(19) United States
(12) Patent Application Publication
(10) Pub. No.: US 2018/0256537 A1
(43) Pub. Date: Sep. 13, 2018

(54) METHODS FOR SURVIVAL AND REJUVENATION OF DERMAL FIBROBLASTS USING PKC ACTIVATORS

(71) Applicants: Tapan K. KHAN, Morgantown, WV (US); Daniel L. ALKON, Chevy Chase, MD (US)

(72) Inventors: Tapan K. KHAN, Morgantown, WV (US); Daniel L. ALKON, Chevy Chase, MD (US)

(21) Appl. No.: 15/762,580

(22) PCT Filed: Sep. 23, 2016
(86) PCT No.: PCT/US2016/053247

§ 371 Date: Mar. 23, 2018

Related U.S. Application Data
(60) Provisional application No. 62/222,701, filed on Sep. 23, 2015.
(60) Provisional application No. 62/356,094, filed on Jun. 29, 2016.

(51) Int. Cl. A61K 31/366; A61K 45/06; A61K 9/00; A61K 9/06; A61P 17/02; A61P 17/10

(52) U.S. Cl. CPC A61K 31/366; A61K 45/06; A61K 9/0014; A61K 8/4973; A61P 17/02; A61P 17/10; A61K 9/06

(57) ABSTRACT

The PKC activator bryostatin-1 and its analogs increase the survival of dermal fibroblast cells. Bryostatin and picolog, a synthetic analog, are used as candidate therapeutic agents for improving the appearance of aging skin, reducing scar tissue formation, and improving the acceptance of a clinical skin grafts.

This application claims priority to U.S. Provisional Application No. 62/222,701 filed Sep. 23, 2015 and to U.S. Provisional Application No. 62/356,094 filed Jun. 29, 2016. The entire contents of both applications are incorporated herein by reference.

The appearance and condition of the skin may be degraded through the effects of naturally occurring environmental factors such as sunlight, wind abrasion, or humidity and through the effects of man-made environmental factors, such as heating, air-conditioning, and pollutants. Pathological processes such as dermatological diseases or the normal aging process also affect the appearance and condition of skin.

The various insults to which the skin is exposed to daily, may act individually or synergistically. To support and improve skin health, consumers have increasingly sought new and improved cosmetic products, such as topical creams, sun care products as well as new, improved cosmetic methods for skin care. Many consumers rely on medicinal products, such as topical antioxidants, cell stimulators, and anti-aging products to treat wrinkles, furrowing of skin and creasing. For some consumers costly; invasive procedures such as surgical restoration, laser wrinkle removal procedures, and hormone replacement therapy are necessary to ameliorate signs of aging.

Injections of biostimulators, compounds that stimulate dermal fibroblast growth and increase their biosynthetic capacity are also used to combat the signs of aging. Illustrative of such compounds are growth factors, growth hormones, vitamins (retinols), and other antioxidant agents. Biostimulators activate fibroblasts to synthesize and secrete structural proteins, such as collagen and extracellular matrix proteins that naturally support the structural integrity of the skin and increase skin elasticity. Thus, skin rejuvenation with injectable biostimulators attempts to mimic the optimal physiological environment for supporting cellular activity of fibroblasts.

Fibroblast proliferation, differentiation, and survival is associated with the activation of an extracellular-signal-regulated kinase (Erk) activity in the MAPK signaling pathway. By contrast, stress-associated c-Jun N-terminal kinase (JNK) and p38 activation are associated with fibroblast growth arrest and apoptosis. In aged skin, the activity of Erk is reduced, while JNK and p38 kinases are increased, compared to young skin (Shin et al, H₂O₂ accumulation by catalase reduction changes MAP kinase signaling in aged human skin in vivo, J. Invest. Dermatol., 125: 221-29, 2005). Photodynamic therapy (PDT) has also been used to rejuvenate aged skin by activating dermal fibroblast Erk. With PDT, prolonged activation of Erk is mediated by an increase in intracellular reactive oxygen species (ROS) (Jang et al, Prolonged activation of ERK contributes to the photorejuvenation effect in photodynamic therapy in human dermal fibroblasts, J. Invest. Dermatol., 133:2265-75, 2013). Yet PDT-generated ROS can also mediate other toxic cellular effects such as lipid peroxidation, direct/indirect cytotoxicity, and production of free radicals leading to oxidative damage (Brackett and Gollnick et al, 2011). Methods for stimulating Erk independent of ROS can avoid these toxic effects and improve skin health. Bryostatin-1 is a macrocyclic lactone that was first isolated from the bryozoan, Bugula neritina (Pettit et al, Isolation and structure of bryostatin 1, J. Am. Chem. Soc., 104: 6846-48, 1982). Bryostatin-1 shows a wide range of biological effects, including anticancer effects (Zhang et al, Preclinical pharmacology of the natural product anticancer agent bryostatin-1, an activator of protein kinase C, Cancer Res., 56: 802-8, 1996), synaptogenic effects that enhance memory (Sun and Alkon, Dual effects of bryostatin-1 on spatial memory and depression, Eur. J. Pharmacol., 512: 43-51, 2005; Kuzirian et al, Bryostatin enhancement of memory in Hermissenda, Biol. Bull., 210: 201-14, 2006), and anti-HIV effects (Perez et al, Bryostatin-1 synergizes with histone deacetylase inhibitors to reactivate HIV-1 from latency, Curr. HIV Res., 8: 418-29, 2010). Bryostatin-1 induces PKC-activated pathways involved in important cellular functions such as cell survival, proliferation, protein synthesis, and apoptosis. Based on the observation that bryostatin-1 activates Erk (Zhao et al, 2002, Khan and Alkon, 2006) the present inventors hypothesize that bryostatin-1 and its synthetic analogs can be used to activate dermal fibroblasts and improve skin health.

This application relates to the use of PKC activators such as bryostatin-1 and its synthetic analog, picolog, to activate Erk. Also described is the use of PKC activators such as bryostatin-1 or this synthetic analogs to increase the survival rate of fibroblasts and enhance the structural integrity of a human skin equivalent (HNEs) composed of primary human fibroblasts and keratinocytes.

The present invention relates to methods for improving one or more signs of aging, as well as to methods for reducing scar tissue formation following injury or surgical intervention in a human in need of such treatment. Also described is a method for promoting wound healing and a method for improving the acceptance of a clinical skin graft using PKC activators as therapeutic agents.

In one embodiment, the PKC activator is the macrocyclic lactone bryostatin. According to this aspect of the invention, the PKC activator is bryostatin-1.

According to another embodiment, the PKC activator is an analog of bryostatin or a bryolog. For example, picolog an exemplary synthetic analog bryostatin-1, is used as the PKC activator in methods described herein.

In one embodiment, the application provides a method of improving the appearance of one or more signs of aging by applying to a skin surface a topical composition comprising a therapeutically effective amount of a PKC activator, or an analog of a PKC activator, and a dermatologically acceptable carrier. According to the disclosed method, the composition is applied for a period of time sufficient to improve the appearance of one or more signs of aging skin. For example, the method improves the appearance of skin by diminishing age spots, reducing wrinkles, stretch marks, acne, and dry skin. In one embodiment, the method improves the appearance of skin by preventing loss of skin elasticity and preventing skin transparency.

The topical composition further comprises one or more additional ingredient chosen water, solvent, preservative, surfactant, gelling agent, and a pH balancer. In one aspect, the topical composition is in the form of a gel or a cream.

According to one embodiment, the PKC activator is chosen from FGF-18, a macrocyclic lactone, benzolactam, a pyrrolidinone, bryolog, a fatty acid derivative, or a diacylglycerol derivative. In one embodiment, the PKC activator is the macrocyclic lactone is bryostatin. Illustrative of the category “bryostatin” are bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

According to another embodiment, the PKC activator is a bryology, for example picolog. A therapeutically effective amount of a PKC activator in the composition ranges from about 0.3×10⁻⁷% by total weight of composition to about 10% by weight of the composition.

According to an aspect of the disclosed method, the PKC activator is provided in a range of about 0.01 nanomoles to about 10 micromoles per unit volume of the topical composition. In one embodiment, the PKC activator is provided in a range of about 0.01 nanomoles to about 1.0 micromoles per unit volume of the topical composition.

According to yet another embodiment, the present disclosure provides a method for decreasing the formation scar tissue following injury to the skin of a human subject, by applying a topical composition to the site of injury. According to the disclosed method, the topical composition is formulated to contain a therapeutically effective amount of a PKC activator, or an analog of a PKC activator, and a dermatologically acceptable carrier. In one embodiment, the composition for decreasing the formation scar tissue further comprises an antibiotic agent.

For certain aspects of the disclosed method, the topical composition is applied to the site of injury over a period of time ranging from about 1 day to about 30 days. The frequency of application can be altered depending on the type of injury, as well as the age and health of the person. In one embodiment, the topical composition is applied once daily, twice daily, thrice daily or four times within a period of 24 hours.

The application also discloses a method for decreasing the formation scar tissue following surgery, by placing a topical composition comprising a therapeutically effective amount of a PKC activator, or an analog of a PKC activator onto a surface of a sterile mesh that is adapted for placement at a site of surgery or around a surgical incision. According to this method, the mesh with the topical composition is placed over or around a surgical incision or a site of anastomosis during surgery or after completing a surgical procedure.

In one embodiment, a composition comprising an analgesic agent is placed on the surface of the sterile mesh along with the composition of a PKC activator. The application also provides in one embodiment a method for healing wounds or promoting wound-healing in a diabetic subject by providing a composition comprising a therapeutically effective amount of a PKC activator, or an analog of a PKC activator to a diabetic subject in need of treatment.

In one embodiment, the composition is formulated as a topical cream or gel and is directly applied to the wound. Such application is continued for a period of time sufficient to heal the wound or promote healing of the wound. In one embodiment, an antibiotic agent is admixed or administered concurrently with a topical cream or gel comprising the PKC activator.

According to another aspect, wound healing is promoted by a composition of a PKC activator, or an analog of a PKC activator by systemic administration of the composition to a diabetic subject.

The application also provides a method for improving the acceptance of a clinical skin graft in a subject in need thereof, by obtaining a donor skin as a graft tissue and contacting the donor skin with a composition comprising a PKC activator, vitamins, amino acids, fibroblast growth factors and hormones for a period of 2-48 hours prior to use, and surgically grafting the donor skin to an area of a body of the subject with skin loss.

According to an aspect of this method, pre-incubation of donor skin with the composition comprising a PKC activator, fibroblast growth factors and hormones reduces the risk of rejection by the subject receiving the graft.

Typically, the composition that is contacted with the donor skin comprises fibroblast growth factors including bovine pituitary extract, human epithelial growth factor, insulin, transferrin, epinephrine, and hydrocortisone. In one embodiment of the method, the donor skin obtained as graft tissue is an autologous graft. According to another embodiment, the donor skin obtained as graft tissue is an allogeneic graft and the subject is administered an immunosuppressive agent following surgical transplantation of the allogeneic graft. Depending on the need, the skin graft is a split-thickness skin graft or a full-thickness skin graft.

### BRIEF DESCRIPTION OF THE FIGURES

FIG. 1: Schematic diagram illustrating the manufacture of human skin equivalents (HSEs).

FIG. 2: Protection of human skin fibroblasts by bryostatin1 (Bry) cultured under long-term serum starvation conditions in 6-well tissue culture plates. (A) Loss of cellular integrity by human fibroblasts cultured under serum starved medium and in the absence of Bryostatin-1

(Bry−); (B) Bryostatin-1 (0.3 nM) maintains cellular integrity by human fibroblasts cultured under serum starved medium (Bry+); (C) Prolonged exposure (20 days) of human fibroblast cells to serum starved medium increase death of these cells; (D). Protection of cultured human skin fibroblasts by bryostatin-1 (Bry+) under prolonged exposure to serum starved medium; (E-G) Bryostatin-1 (0.3 nM), (Bry+), protects skin fibroblasts cultured on a collagen matrix from serum starvation over a prolonged time period of 25 days; (H-J) Serum starvation over a prolonged time period of 25 days results in a loss of cellular integrity of skin fibroblast cells cultured on a collagen matrix (Bry−). Images are representative of experiments conducted in triplicate. A-D: Bar=25 μm, and E-J Bar=10 μm.

FIG. 3: Picolog, a synthetic analog of bryostatin-1, protects cultured human skin fibroblasts from long-term serum starvation. (A) Loss of cellular integrity of fibroblasts after serum starvation over a period of 15 days (no treatment; (Picolog-)). (B) Treatment with picolog (5 nM) maintains fibroblast cellular integrity following serum starvation over a period of 15 days. (C) Viability of cultured fibroblasts following serum starvation over a period of 15 days was greater in the presence of picolog compared to untreated control cultures of fibroblasts. Data are presented in terms of relative cell viability. Results are mean±SEM for 15 wells. a-b: Bar=25 μm.

FIG. 4A, Left Panel: Bryostatin activates Erk and protects dermal fibroblasts. Loss of cellular integrity and greater cell death of untreated dermal fibroblasts maintained in a serum deprived culture for 5 days (Bry− Inh−). Treatment with Bryostatin-1 improves cell integrity and promotes survival of dermal fibroblast cells maintained in serum deprived culture for 5 days (Bry+ Inh−).

FIG. 4A, Right Panel: Treatment of dermal fibroblast cultured in serum deprived medium with the Erk inhibitor PD98059 (Bry− Inh+), results in cell death and loss of cellular integrity similar to untreated cells. Treatment with Bryostatin does not reverse the effects of Erk inhibition (Bry+ Inh+). Images are representative of experiments conducted in triplicate.

FIG. 4B: Immunoblot analysis to measure activation of Erk in response to 0.3 nM bryostatin-1 in serum-deprived cultured human skin fibroblasts. Bryostatin-1 induced prolonged activation of Erk.

FIG. 4C: Bar graph illustrating quantification of activated Erk1/2 (p-Erk1/2) by normalizing against total Erk1/2 (p-Erk/Erk). Results are mean±SEM from three independent measurements starting from cell culturing.

FIG. 4D: Immunoblot illustrating the activation of Erk in fibroblasts treated with bryostatin-1 alone (Bry), or a combination of Bryostatin-1 and the Erk inhibitor PD98059 (Inh; 30 μM). PD98059 blocks bryostatin-induced Erk activation in cultured human fibroblasts. Results are mean±SEM for three different experiments; Bar=25 μm.

FIG. 5: Prolonged treatment by bryostatin-1 reduces apoptosis and protects dermal fibroblasts. (A) Dermal fibroblasts were cultured for 24 hours in serum deprived medium before treatment with 0.3 nM bryostatin-1. Micrographs at day 35 shows greater cell viability and cellular integrity for bryostain-1 treated fibroblasts compared to untreated fibroblasts. Addition of 10% serum to the culture medium on day 35 further improves cellular integrity for bryostain-1 treated fibroblasts compared to untreated fibroblasts. (B, C) Prolong treatment of dermal fibroblasts in culture with bryostatin-1 reduces caspase-8 expression levels. The reduction of caspase-8 expression is greater after 3 days of treatment with bryostatin-1, (5 measurements in cells isolated from three individuals).

FIG. 6: Bryostatin1 promotes cell survival within human skin equivalents (HSE's). (A) Single treatment of HSE's with bryostatin-1 (0.3 nM) (Bry+) shows less cell death than untreated HSE's (Bry−). (B) HSE's treated with multiple doses of bryostatin1 (0.03 nM; Bry+) showed greater cell viability than HSEs treated with a single 0.3 nM dose of Bryostatin-1 or untreated HSE's (Bry−). Images are representative of experiments conducted in triplicate. A: Bar=100 μm, B: Bar=50 μm.

FIG. 7: Schematic illustrating two possible pathways for activating Erk in skin fibroblasts. Pathway I: Use of growth factor(s) to activate Erk; Pathway II: Bryostatin induced activation of Erk.

### DESCRIPTION

As used herein, the singular forms “a,” “an,” and “the” include plural reference. As used herein, “protein kinase C activator” or “PKC activator” refers to a substance that increases the rate of the reaction catalyzed by PKC. PKC activators can be non-specific or specific activators. For example, a specific activator activates one PKC isoform, e.g., PKC-ε (epsilon), to a greater detectable extent than another PKC isoform.

As used herein, the term “fatty acid” refers to a compound composed of a hydrocarbon chain and ending in a free acid, an acid salt, or an ester. When not specified, the term “fatty acid” is meant to encompass all three forms. Those skilled in the art understand that certain expressions are interchangeable. For example, “methyl ester of linolenic acid” is the same as “linolenic acid methyl ester,” which is the same as “linolenic acid in the methyl ester form.”

Illustrative PKC activators suitable for use with the disclosed method include macrocyclic lactones, bryologs, picolog, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

The term “picolog” refers to an analog of bryostatin that has the following chemical structure:

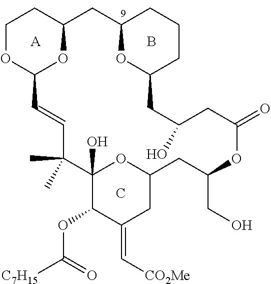

As used herein, the term “cyclopropanated” or “CP” refers to a compound wherein at least one carbon-carbon double bond in the molecule has been replaced with a cyclopropane group. The cyclopropyl group may be in cis or trans configuration. Unless otherwise indicated, it should be understood that the cyclopropyl group is in the cis configuration. Compounds with multiple carbon-carbon double bonds have many cyclopropanated forms. For example, a polyunsaturated compound in which only one double bond has been cyclopropanated would be said to be in “CP1 form.” Similarly, “CP6 form” indicates that six double bonds are cyclopropanated.

For example, docosahexaenoic acid (“DHA”) methyl ester has six carbon-carbon double bonds and thus can have one to six cyclopropane rings. Shown below are the CP1 and CP6 forms. With respect to compounds that are not completely cyclopropanated (e.g. DHA-CP1), the cyclopropane group(s) can occur at any of the carbon-carbon double bonds.

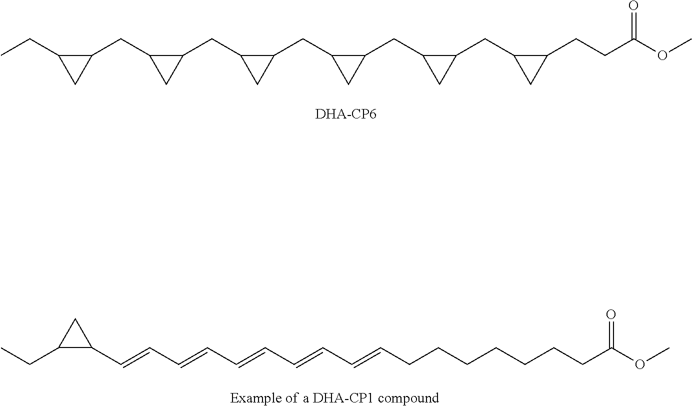

The phrase “pharmaceutically acceptable” refers to molecular entities and compositions that are physiologically tolerable and do not typically produce untoward reactions when administered to a subject. For example, as used herein, the term “pharmaceutically acceptable” means approved by a regulatory agency of the federal or a state government or listed in the U.S. Pharmacopeia or other generally recognized pharmacopeia for use in animals, and more particularly in humans. The term “pharmaceutically acceptable carrier” means a chemical composition with which the active ingredient may be combined and which, following the combination, can be used to administer the active ingredient to a subject and can refer to a diluent, adjuvant, excipient, or vehicle with which the compound is administered.

The terms “therapeutically effective dose” and “effective amount” refer to an amount of a therapeutic agent that results in a measurable therapeutic response. A therapeutic response may be any response that a user (e.g., a clinician) will recognize as an effective response to the therapy, including improvement of symptoms and surrogate clinical markers. Thus, a therapeutic response will generally be an amelioration or inhibition of one or more symptoms of a disease or condition. A measurable therapeutic response also includes a finding that a symptom or disease is prevented or has a delayed onset, or is otherwise attenuated by the therapeutic agent.

The terms “approximately” and “about” mean to be nearly the same as a referenced number or value including an acceptable degree of error for the quantity measured given the nature or precision of the measurements. As used herein, the terms “approximately” and “about” should be generally understood to encompass ±20% of a specified amount, frequency or value. Numerical quantities given herein are approximate unless stated otherwise, meaning that term “about” or “approximately” can be inferred when not expressly stated.

The terms “administer,” “administration,” or “administering” as used herein refer to (1) providing, giving, dosing and/or prescribing by either a health practitioner or his authorized agent or under his direction a composition according to the disclosure, and (2) putting into, taking or consuming by the patient or person himself or herself, a composition according to the disclosure. As used herein, “administration” of a composition includes any route of administration, including oral, intravenous, subcutaneous, intraperitoneal, and intramuscular.

Skin aging is a multifactorial process driven by both intrinsic and extrinsic factors. For example, intrinsic factors include chronological aging and other biochemical changes occurring in skin cells. Extrinsic factors, e.g., include UV exposure, toxins, pollutants, wind, heat, low humidity, harsh surfactants, abrasives, smoke and other environmental elements.

The effects of aging can result in a number of visible changes in the appearance of skin. Thinning of the stratum corneum layer of the epidermis and general degradation of the skin by intrinsic and/or extrinsic factors increases the visible appearance of fine lines, wrinkles, inflammation, uneven skin tone and other signs of skin aging. For example, in young skin, melanin is evenly distributed, but as skin ages or is exposed to damaging environmental effects, melanocytes lose their normal regulation process and produce excess pigment, leading to areas of hyperpigmentation such as age spots (lentigines) and uneven skin tone.

The present disclosure relates to methods for improving the appearance of aging skin by applying a topical composition containing a therapeutically effective amount of a PKC activator or an analog of a PKC activator and a dermatologically acceptable carrier. PKC's are a family of proteins that control the function of other cellular important proteins. PKC's are implicated to play a role in skin health.

### PKC Activators Protect Human Skin Equivalents (HSEs) and Human Skin Fibroblasts Under Stress Conditions

Human skin equivalents (HSEs) are a bioengineered combination of primary human skin cells, such as keratinocytes and dermal fibroblasts supported on an extracellular matrix of collagen. HSE's are used in vitro as models of human skin to test the efficacy of drugs formulated to treat diseased skin conditions as well as to test the efficacy of drugs at enhancing wound healing. Advanced Skin Test 2000 (AST2000) developed by Cell Systems Biotechnologie GmbH is a product consisting of embedded fibroblasts as base and an epidermal layer of keratinocytes for testing in in vitro skin permeation and toxicology screening models.

HSEs are also used clinically as skin grafts. In fact, human epidermal keratinocytes and dermal fibroblasts cultured in separate layers on a Type I bovine collagen sponge by FortiCell Bioscience® has been approved by FDA for use as a skin graft.

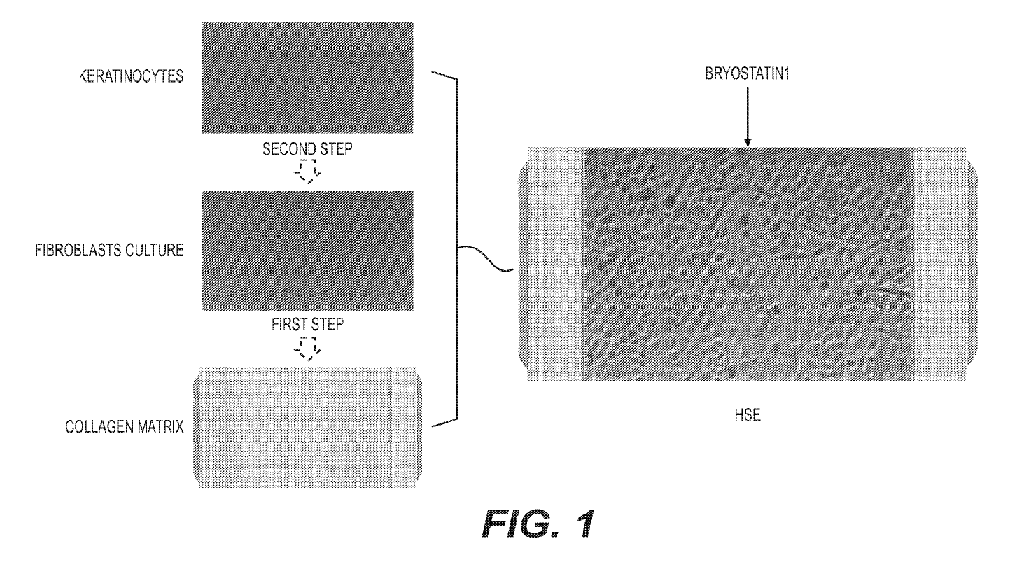

FIG. 1 illustrates a model human skin equivalent used by the present inventors to test the effect of bryostatin on fibroblast survival and viability. This HSE was manufactured by creating a dermal matrix formed of skin fibroblast cells cultured on collagen matrix and seeding the top of the dermal matrix with human keratinocytes to form an epidermal layer. HSEs were treated with vehicle (control) or bryostatin-1 (test group) and cell structure, growth and cell viability were monitored by microscopic observation as a function of time.

FIG. 6A (Bry+ column) illustrates micrographs of the test and control HSEs. HSEs treated with bryostatin-1 showed fewer dead fibroblast cells overall, demonstrating that bryostatin-1 protects fibroblasts in HSEs. In contrast, untreated HSEs show significant fibroblast cell death in culture (FIG. 6A, Bry− column). FIG. 6 further illustrates that the protective effect of bryostatin-1 decreases as a function of time. For example, fewer dead fibroblast cells were observed in the bryostatin-1 treated HSEs after 9 days in culture than after 12 days in culture. For the untreated HSEs, however, significant death of fibroblast cells was observed as early as 6 days in culture with a complete breakdown of cell structure and integrity at 12 days.

The frequency of treatment with a PKC activator such as bryostatin-1, affects viability of cultured HSE fibroblasts. As FIG. 6B illustrates, the survival of fibroblast cells in HSEs receiving two doses of bryostatin-1 (0.3 nm) was greater than fibroblasts in HSEs receiving a single 0.3 nM dose of bryostatin-1. Fibroblast survival and viability was significantly greater in the single dose and two dose treated HSEs compared to untreated HSEs (FIG. 6b; panel: Bry−), supporting bryostatin-1's role in protecting fibroblast cells.

The processes that cause skin to age are complex. The slowing of epidermal and dermal turnover rate, and gradual loss of skin elasticity due to degradation of collagen fibers and structural proteins are two processes that accompany skin aging. Bryostatin-1 was found by the present inventors to slow skin aging by activating fibroblasts to secrete structural proteins and collagen fibers necessary to maintain skin elasticity, promote wound healing and reduce the appearance of wrinkles and age spots.

In one embodiment the disclosure provides a method for improving the appearance of aging skin by applying to a skin surface a composition of a PKC activator. In one embodiment, the composition is formulated as a topical cream or a gel containing a PKC activator and a pharmaceutically acceptable dermal carrier.

In one embodiment, the PKC activator in a composition to improve the appearance of aging skin according to the disclosed method is a macrocyclic lactone or a bryolog. According to one aspect, the PKC activator is bryostatin-1 or its synthetic analog “picolog”.

As further described below, both bryostatin-1 and picolog reduced stress associated with the growth of fibroblast cells for extended periods of time under serum deprived conditions. Treatment with bryostatin-1 or picolog permitted growth of primary cultures of human fibroblasts under serum deprived conditions over extended periods of time. In contrast, serum deprivation is stressful and impacts the structural integrity and viability of fibroblast cells in culture. Significant loss of fibroblast cells was observed in cultures that did not contain bryostatin-1 or picolog. The present inventors surprisingly discovered that sub-nanomolar concentrations of bryostatin-1 or picolog are needed to protect fibroblast cells from stress associated with growth under serum deprived conditions.

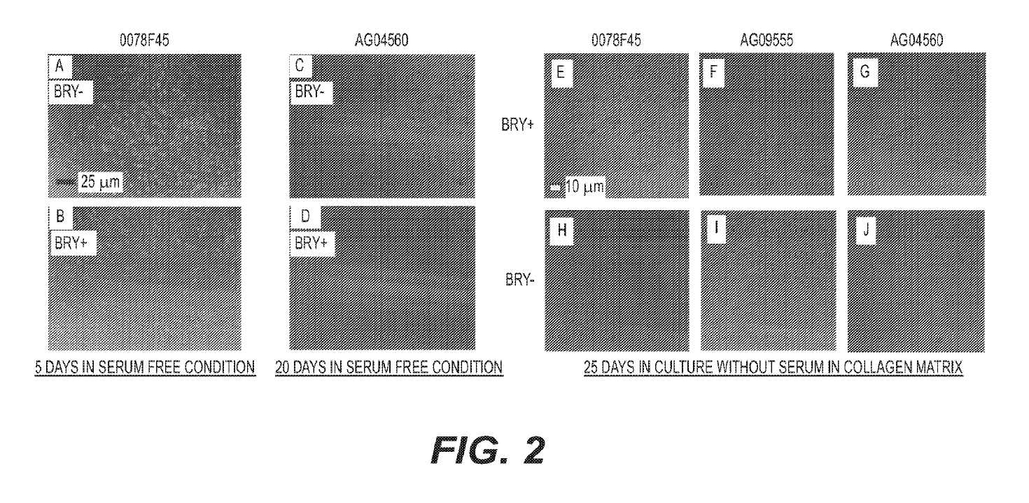

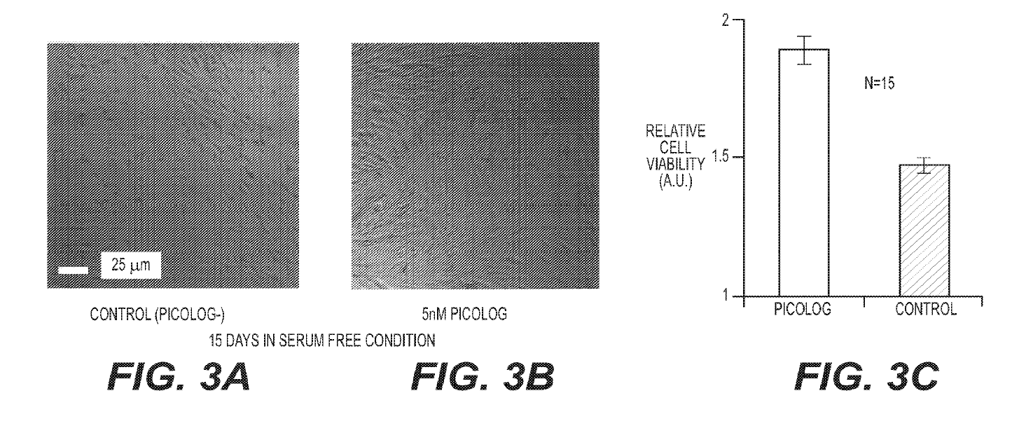

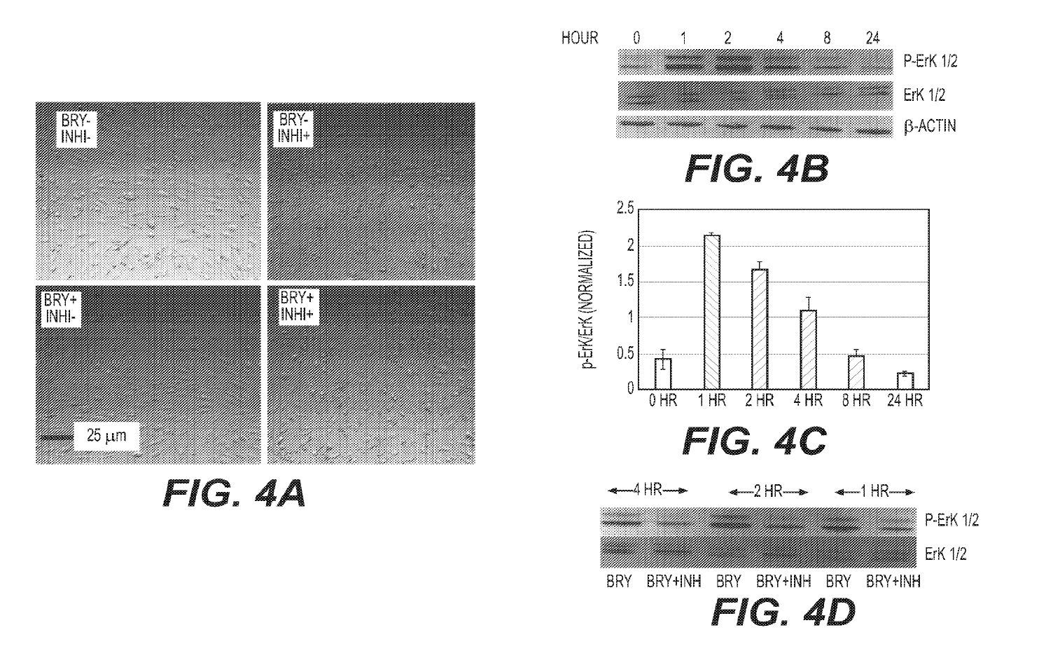

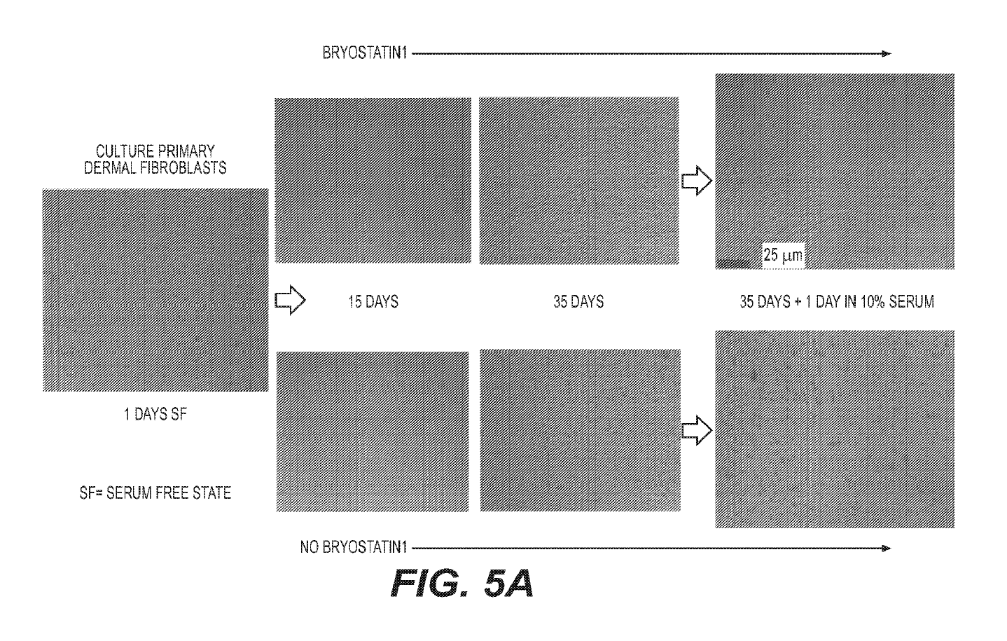

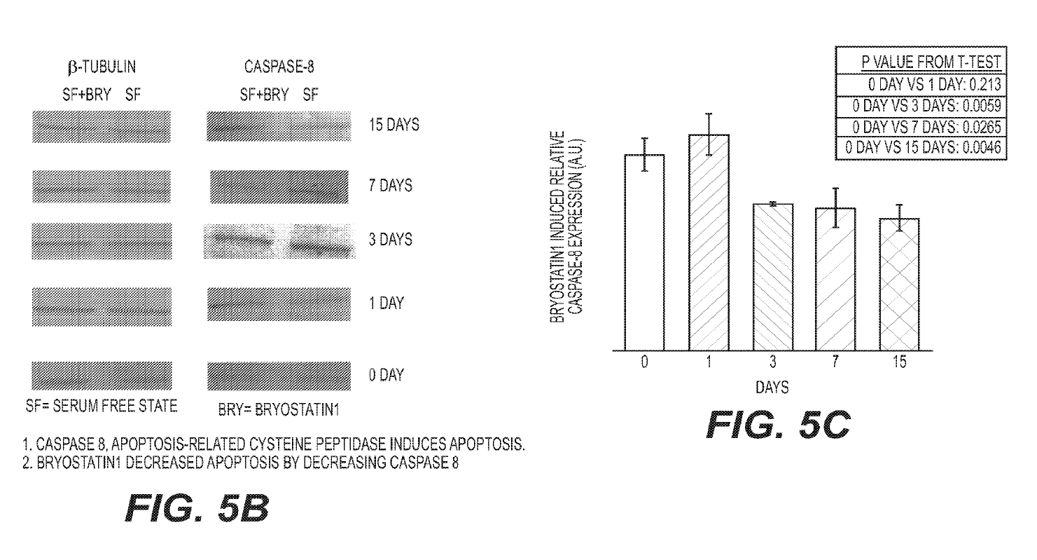

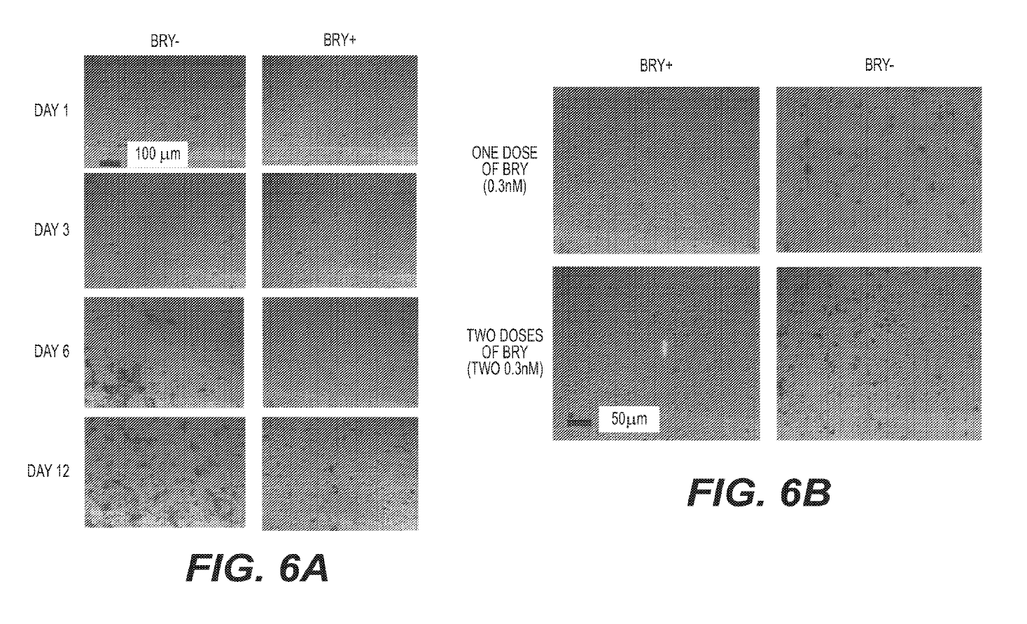

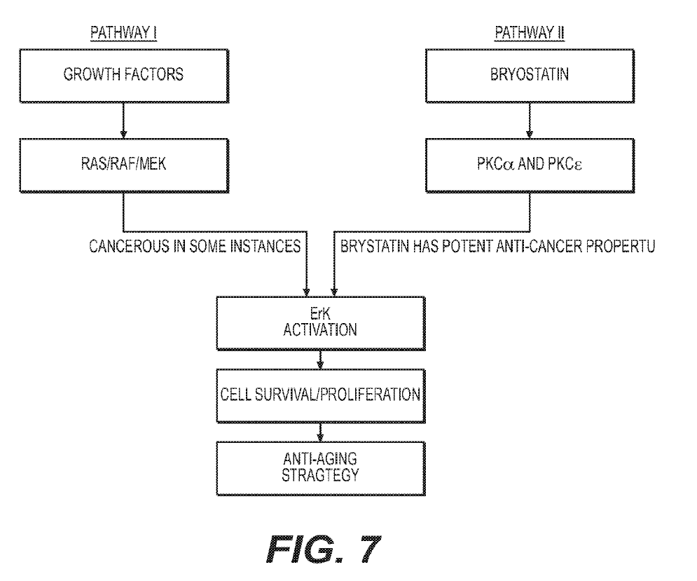

As illustrated in FIG. 2, bryostatin-1 protects freshly isolated human skin fibroblasts cultured in serum starved medium Skin fibroblast cells were isolated from a skin punch biopsy and cultured in 6-well tissue culture plates. After culturing cells for 24 hours in serum starved medium, cells in the test wells of the tissue culture plate were treated with bryostain-1 (final concentration 0.3 nM/well; Bry+), while control wells received a vehicle (Bry−; FIG. 2A, 2B). Fibroblast cells in the test and control wells were permitted to grow for an additional 5 days under serum-free conditions, prior to visualization microscopically. FIG. 2A illustrates that fibroblasts in the control (Bry−) group lack structural integrity. In contrast, fibroblasts cultured in the presence of bryostatin-1 are healthy and appear to be normal (FIG. 2B). Cell viability studies further showed a lower percentage of viable fibroblast in the control group compared to the bryostatin-1 treated group.

Similar results were observed for banked skin fibroblast cells. Bryostatin-1 protected fibroblast cells from the trauma associated with prolonged exposure to serum starved conditions, such as 20 days. As FIGS. 2C and 2D show, in the absence of bryostatin-1 there is a total loss of cellular integrity under serum free conditions. Meanwhile, the addition of sub-nanomolar amounts of bryostatin-1 to the culture medium prevented the death of human skin fibroblasts in serum free cultures.

Bryostain-1 elicits a similar protective effect on collagen matrix supported human skin fibroblast cells under serum starved conditions. As FIGS. 2E-2J show bryostatin-1 maintains the cellular integrity of cultured fibroblast cells for 25 days under serum-starved conditions (Bry+; FIGS. 2E-2G). The absence of bryostatin-1 from the culture medium caused a loss of cellular structure and cell viability as fibroblasts are stressed under serum starved conditions (Bry−; FIGS. 2H-2J).

Picolog also exerts a similar protective effect. As illustrated by the microscopic images in FIGS. 3A and 3B, the addition of picolog at a concentration of 5 nM to a culture of human skin fibroblast cells increased cell survival by protecting the fibroblast cells under serum free condition over a period of 15 days. Picolog also increased the relative viability of fibroblast cells compared to untreated cells (FIG. 3C). Together, these results suggest that bryostatin-1 and picolog protect human skin fibroblast from trauma associated with growth in a serum deprived medium.

Dermal fibroblasts are the primary cells responsible for structure, appearance and elasticity of human skin. Dermal fibroblast synthesize collagen fibers that forms the basic structural scaffold of skin. In addition, these cells produce structural proteins such as elastin and glycosaminoglycans (GAGs), which are necessary for skin elasticity. As described above, bryostatin-1 and picolog promote the survival of human fibroblasts over extended periods of time under serum deprived conditions. However, the mechanism for the observed protective effect by these compounds is not well known.

The present inventors decided to investigate the role of PKC activators in protection of human skin fibroblasts. The inventors found that PKC activators exert cell protective effects by activating Erk signaling pathways in dermal fibroblast cells. Specifically, it was observed that the protective effect was due to the ability of bryostatin-1 and picolog to increase Erk-mediated biosynthesis of structural proteins and collagen in dermal fibroblast cells.

Further support for the observation that bryostatin-1 activates Erk signaling in dermal fibroblasts stems from immunoblot analysis for Erk in fibroblast cells treated with bryostatin1 for various lengths of time and cultured under serum-starved conditions. As illustrated by FIGS. 4B and 4C, bryostatin1 induced activation of Erk1/2 in fibroblasts cultured under serum-free conditions.

The addition of an Erk inhibitor PD98059, to fibroblasts cultured in the presence of bryostatin-1 under serum-free conditions lowered the cell protective effects of bryostatin-1. Indeed, as FIG. 4A illustrates, cells treated with bryostatin-1 alone showed the greatest viability and good cellular integrity (FIG. 4a; panel: Bry+ Inhi−), compared to cells exposed to the Erk inhibitor alone, or cells exposed to a combination of bryostatin-1 and PD98059 (FIG. 4a, panel Bry−Inhi+, Bry+ Inhi+). The loss of cell integrity and evidence of cell death in cultures containing the Erk inhibitor alone or the combination of the Erk inhibitor and bryostatin-1 was comparable to cultures of untreated fibroblast. Immunoblot analysis for Erk using fibroblast cells exposed to bryostatin-1 and the Erk inhibitor are shown in FIG. 4D. It is clear, that the Erk activating effect of bryostatin-1 is significantly blocked by PD98059, at least when present at a concentration of 30 μM.

Supportive of the role of bryostatin-1 or picolog in maintaining the integrity of skin fibroblast cells under stressful conditions is the observation that bryostatin-1 inhibits the production of matrix metalloproteins 1, 3, 9, 10 and 11 (MMP's), implicated to play a role in degrading the extracellular matrix of skin. MMP's weaken skin structure, degrade skin tone and enhance the appearance of aging, primarily by degrading fibrillar and non-fibrillar collagens, fibronectin, laminin, and glycoproteins present in the basement membrane of skin.

Since collagen and structural proteins such as elastin are important for cell integrity and for maintaining skin health, up-regulating Erk-mediated biosynthesis of these proteins by PKC activators of the invention provides one method for improving the appearance of aging skin. Thus, topical compositions of one or more PKC activators in a suitable dermal carrier can be used to improve aging skin, and decrease scar tissue formation according to the disclosed method. In one embodiment, PKC activators are macrocyclic lactones, e.g., the bryostatin and neristatin classes, which act to stimulate PKC. Macrocyclic lactones (also known as macrolides) generally comprise 14-, 15-, or 16-membered lactone rings. Macrolides belong to the polyketide class of natural products. Macrocyclic lactones and derivatives thereof are described, for example, in U.S. Pat. Nos. 6,187,568; 6,043,270; 5,393,897; 5,072,004; 5,196,447; 4,833,257; and U.S. Pat. Nos. 4,611,066; and 4,560,774; each incorporated by reference herein in its entirety. Those patents describe various compounds and various uses for macrocyclic lactones including their use as an anti-inflammatory or anti-tumor agent. See also Szallasi et al. J. Biol. Chem. (1994), vol. 269, pp. 2118-2124; Zhang et al., Cancer Res. (1996), vol. 56, pp. 802-808; Hennings et al. Carcinogenesis (1987), vol. 8, pp. 1343-1346; Varterasian et al. Clin. Cancer Res. (2000), vol. 6, pp. 825-828; Mutter et al. Bioorganic & Med. Chem. (2000), vol. 8, pp. 1841-1860; each incorporated by reference herein in its entirety.

Of the bryostatin class of compounds, bryostatin-1 is particularly interesting. It has been shown to activate PKC without tumor promotion. Further, its dose response curve is biphasic. In addition, bryostatin-1 demonstrates differential regulation of PKC isoforms including PKC-α, PKC-δ and PKC-ε. Given this potential, bryostatin-1 has undergone toxicity and safety studies in animals and humans, and is actively being investigated as an anti-cancer agent as an adjuvant with other potential anti-cancer agents.

Bryostatins as a class are thought to bind to the C1a site (one of the DAG binding sites) and cause translocation like a phorbol ester, but unlike the phorbol esters, does not promote tumors. Bryostatin-1 exhibits no toxicity at 20 μg/week, although the use of more than 35 μg/week may be associated with muscle pain. In rats, the acute LD₅₀ value for Bryostatin-1 is 68 μg/kg, and the acute LD₁₀ value is 45 μg/kg. Death in high doses results from hemorrhage.

Bryostatin crosses the blood-brain barrier and is slowly eliminated from the brain, exhibiting slow dissociation kinetics (t_{1/2}>12 hr). In the blood stream, bryostatin has a short half-life (t_{1/2}=1 hr). However, of an initial dose (via intravenous injection), 1% is in the blood at 100 hrs and is detectable in the blood for 14 days after a single injection. Bryostatin tends to accumulate in fatty tissues and is likely detoxified though glycolysation of OH groups and other well-known pathways for detoxification of xenobiotic compounds.

In one embodiment of the present disclosure, the macrocyclic lactone is a bryostatin. Bryostatins include, for example, bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, and bryostatin-18.

In at least one embodiment, the bryostatin is bryostatin-1 whose structure is shown below.

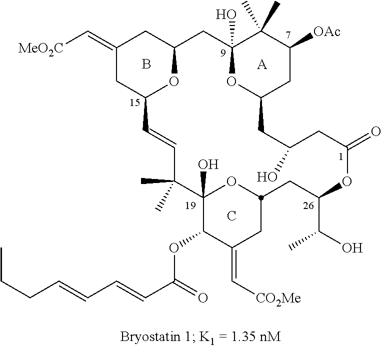

In another embodiment, the bryostatin is bryostatin-2 (shown below; R═COC₇H₁₁, R′═H).

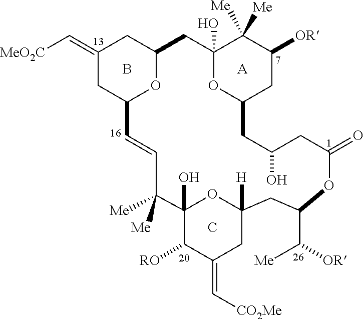

In one embodiment of the present disclosure, the macrocyclic lactone is a neristatin. In one embodiment, the neristatin is chosen from neristatin-1. In another embodiment, the macrocyclic lactone is chosen from macrocylic derivatives of cyclopropanated PUFAs such as, 24-octaheptacyclononacosan-25-one (cyclic DHA-CP6) (shown below).

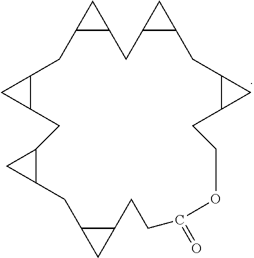 [SMILES: O=C1CCC2CC2CC2CC2CC2CC2CC2CC2CC2CC2CC2CC2CCO1]

In another embodiment, the macrocyclic lactone is a bryolog. Bryologs (analogs of bryostatin) are another class of PKC activators that are suitable for use in the present disclosure. Bryologs can be chemically synthesized or produced by certain bacteria. Different bryologs exist that modify, for example, the rings A, B, and C (see bryostatin-1, shown above) as well as the various substituents. As a general overview, bryologs are considered less specific and less potent than bryostatin but are easier to prepare. It was found that the C-ring is important for binding to PKC while the A-ring is important for non-tumorigenesis. Further, the hydrophobic tail appears to be important for membrane binding.

Table 1 summarizes structural characteristics of several bryologs and demonstrates variability in their affinity for PKC (ranging from 0.25 nM to 10 μM). Structurally, they are all similar. While bryostatin-1 has two pyran rings and one 6-membered cyclic acetal, in most bryologs one of the pyrans of bryostatin-1 is replaced with a second 6-membered acetal ring. This modification reduces the stability of bryologs, relative to bryostatin-1, for example, in both strong acid or base, but has little significance at physiological pH. Bryologs also have a lower molecular weight (ranging from about 600 g/mol to 755 g/mol), as compared to bryostatin-1 (988), a property which facilitates transport across the blood-brain barrier.

TABLE 1
Bryologs.

| Name | PKC Affin (nM) | MW | Description |
|---|---|---|---|
| Bryostatin-1 | 1.35 | 988 | 2 pyran + 1 cyclic acetal + |
| | | | macrocycle |
| Analog 1 | 0.25 | 737 | 1 pyran + 2 cyclic acetal + |
| | | | macrocycle |
| Analog 2 | 6.50 | 723 | 1 pyran + 2 cyclic acetal + |
| | | | macrocycle |
| Analog 7a | — | 642 | 1 pyran + 2 cyclic acetals + |
| | | | macrocycle |
| Analog 7b | 297 | 711 | 1 pyran + 2 cyclic acetals + |
| | | | macrocycle |
| Analog 7c | 3.4 | 726 | 1 pyran + 2 cyclic acetals + |
| | | | macrocycle |
| Analog 7d | 10000 | 745 | 1 pyran + 2 cyclic acetals + |
| | | | macrocycle, acetylated |
| Analog 8 | 8.3 | 754 | 2 cyclic acetals + macrocycle |
| Analog 9 | 10000 | 599 | 2 cyclic acetals |

Analog 1 exhibits the highest affinity for PKC. Wender et al., Curr. Drug Discov. Technol. (2004), vol. 1, pp. 1-11; Wender et al. Proc. Natl. Acad. Sci. (1998), vol. 95, pp. 6624-6629; Wender et al., J. Am. Chem. Soc. (2002), vol. 124, pp. 13648-13649, each incorporated by reference herein in their entireties. Only Analog 1 exhibits a higher affinity for PKC than bryostatin-1. Analog 2, which lacks the A ring of bryostatin-1, is the simplest analog that maintains high affinity for PKC. In addition to the active bryologs, Analog 7d, which is acetylated at position 26, has virtually no affinity for PKC.

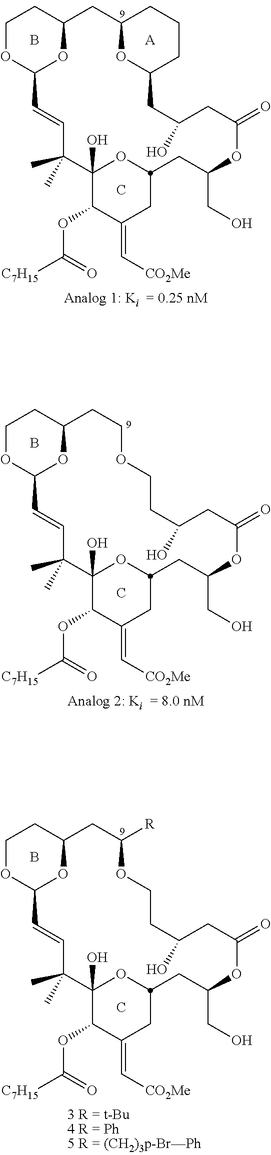

B-ring bryologs may also be used in the present disclosure. These synthetic bryologs have affinities in the low nanomolar range. Wender et aI., Org Lett. (2006), vol. 8, pp. 5299-5302, incorporated by reference herein in its entirety. B-ring bryologs have the advantage of being completely synthetic, and do not require purification from a natural source.

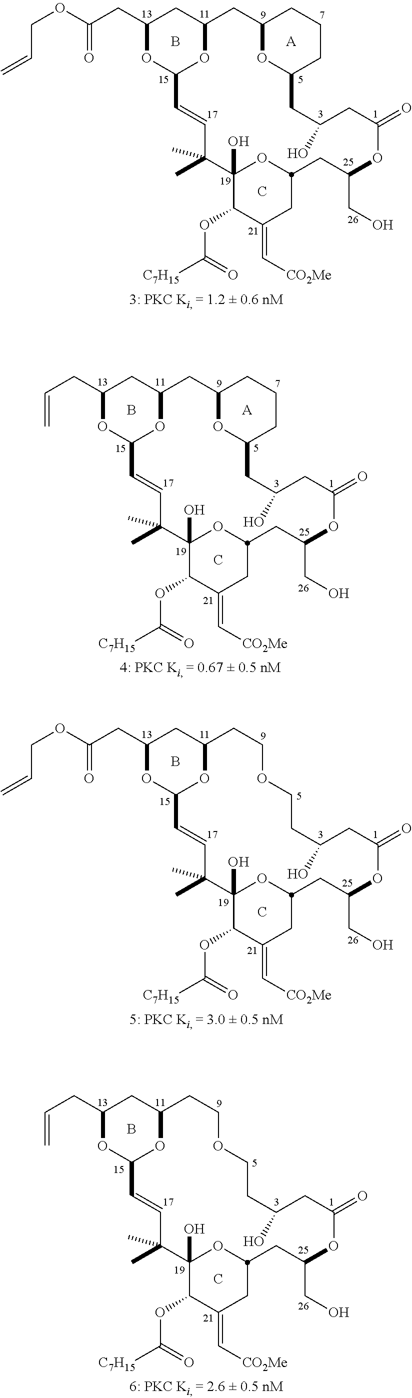

A third class of suitable bryostatin analogs are the A-ring bryologs. These bryologs have slightly lower affinity for PKC than bryostatin-1 (6.5 nM, 2.3 nM, and 1.9 nM for bryologs 3, 4, and 5, respectively) and a lower molecular weight. A-ring substituents are important for non-tumorigenesis.

Bryostatin analogs are described, for example, in U.S. Pat. Nos. 6,624,189 and 7,256,286. Methods using macrocyclic lactones to improve cognitive ability are also described in U.S. Pat. No. 6,825,229 B2.

In certain embodiments, the analog of bryostatin is the synthetic compound picolog that is effective at activating Erk-mediated biosynthesis of structural proteins and collagen in dermal fibroblasts at concentrations in the low nanomolar to sub-nanomolar range.

Another class of PKC activators is derivatives of diacylglycerols that bind to and activate PKC. See, e.g., Niedel et al., Proc. Natl. Acad. Sci. (1983), vol. 80, pp. 36-40; Mori et al., J. Biochem. (1982), vol. 91, pp. 427-431; Kaibuchi et al., J. Biol. Chem. (1983), vol. 258, pp. 6701-6704. Activation of PKC by diacylglycerols is transient, because they are rapidly metabolized by diacylglycerol kinase and lipase. Bishop et al. J. Biol. Chem. (1986), vol. 261, pp. 6993-7000; Chuang et al. Am. J. Physiol. (1993), vol. 265, pp. C927-C933; incorporated by reference herein in their entireties. The fatty acid substitution on the diacylglycerols derivatives determines the strength of activation. Diacylglycerols having an unsaturated fatty acid are most active. The stereoisomeric configuration is important; fatty acids with a 1,2-sn configuration are active while 2,3-sn-diacylglycerols and 1,3-diacylglycerols do not bind to PKC. Cis-unsaturated fatty acids may be synergistic with diacylglycerols. In at least one embodiment, the term “PKC activator” expressly excludes DAG or DAG derivatives.

Another class of PKC activators is isoprenoids. Farnesyl thiotriazole, for example, is a synthetic isoprenoid that activates PKC with a K_{d} of 2.5 μM. Farnesyl thiotriazole, for example, is equipotent with dioleoylglycerol, but does not possess hydrolyzable esters of fatty acids. Gilbert et al., Biochemistry (1995), vol. 34, pp. 3916-3920; incorporated by reference herein in its entirety. Farnesyl thiotriazole and related compounds represent a stable, persistent PKC activator. Because of its low molecular weight (305.5 g/mol) and absence of charged groups, farnesyl thiotriazole would be expected to readily cross the blood-brain barrier.

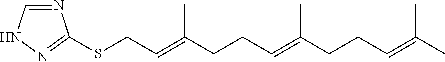 [SMILES: CC(C)=CCC/C(C)=C/CC/C(C)=C/CSc1nc[nH]n1]

Yet another class of activators includes octylindolactam V, gnidimacrin, and ingenol. Octylindolactam V is a non-phorbol protein kinase C activator related to teleocidin. The advantages of octylindolactam V (specifically the (−)-enantiomer) include greater metabolic stability, high potency (EC₅₀=29 nM) and low molecular weight that facilitates transport across the blood brain barrier. Fujiki et al. Adv. Cancer Res. (1987), vol. 49 pp. 223-264; Collins et al. Biochem. Biophys. Res. Commun. (1982), vol. 104, pp. 1159-4166, each incorporated by reference herein in its entirety.

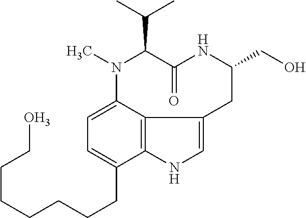 [SMILES: CC(C)[C@H]1C(=O)N[C@H](CO)Cc2c[nH]c3c(CCCCCCCO)ccc(c23)N1C]

Gnidimacrin is a daphnane-type diterpene that displays potent antitumor activity at concentrations of 0.1 nM-1 nM against murine leukemias and solid tumors. It acts as a PKC activator at a concentration of 0.3 nM in K562 cells, and regulates cell cycle progression at the G1/S phase through the suppression of Cdc25A and subsequent inhibition of cyclin dependent kinase 2 (Cdk2) (100% inhibition achieved at 5 ng/ml). Gnidimacrin is a heterocyclic natural product similar to Bryostatin-1, but somewhat smaller (MW=774.9 g/mol).

Iripallidal is a bicyclic triterpenoid isolated from Iris pallida. Iripallidal displays anti-proliferative activity in a NCI 60 cell line screen with GI₅₀ (concentration required to inhibit growth by 50%) values from micromolar to nanomolar range. It binds to PKCα with high affinity (Kᵢ=75.6 nM). It induces phosphorylation of Erk1/2 in a RasGRP3-dependent manner. Its molecular weight is 486.7 g/mol. Iripallidal is about half the size of Bryostatin-1 and lacks charged groups.

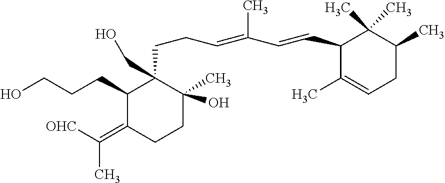

Ingenol is a diterpenoid related to phorbol but less toxic. It is derived from the milkweed plant Euphorbia peplus. Ingenol 3,20-dibenzoate, for example, competes with [3H] phorbol dibutyrate for binding to PKC (Kᵢ=240 nM). Winkler et al., J. Org. Chem. (1995), vol. 60, pp. 1381-1390, incorporated by reference herein. Ingenol-3-angelate exhibits antitumor activity against squamous cell carcinoma and melanoma when used topically. Ogbourne et al. Anticancer Drugs (2007), vol. 18, pp. 357-362, incorporated by reference herein.

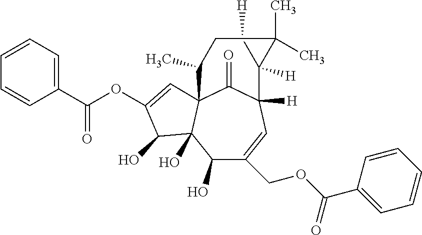

Another class of PKC activators is napthalenesulfonamides, including N-(n-heptyl)-5-chloro-1-naphthalenesulfonamide (SC-10) and N-(6-phenylhexyl)-5-chloro-1-naphthalene sulfonamide. SC-10 activates PKC in a calcium-dependent manner, using a mechanism similar to that of phosphatidylserine. Ito et al., Biochemistry (1986), vol. 25, pp. 4179-4184, incorporated by reference herein. Naphthalenesulfonamides act by a different mechanism than bryostatin and may show a synergistic effect with bryostatin or member of another class of PKC activators. Structurally, naphthalenesulfonamides are similar to the calmodulin (CaM) antagonist W-7, but are reported to have no effect on CaM kinase.

Yet another class of PKC activators is diacylglycerol kinase inhibitors, which indirectly activate PKC. Examples of diacylglycerol kinase inhibitors include, but are not limited to, 6-(2-(4-[(4-fluorophenyl)phenylmethylene]-1-piperidinyl)ethyl)-7-methyl-5H-thiazolo[3,2-a]pyrimidin-5-one (R59022) and [3-[2-[4-(bis-(4-fluorophenyl)methylene]piperidin-1-yl)ethyl]-2,3-dihydro-2-thioxo-4(1H)-quinazolinone (R59949).

Still another class of PKC activators is growth factors, such as fibroblast growth factor 18 (FGF-18) and insulin growth factor, which function through the PKC pathway. FGF-18 expression is up-regulated in learning, and receptors for insulin growth factor have been implicated in learning. Activation of the PKC signaling pathway by these or other growth factors offers an additional potential means of activating PKC.

Another class of PKC activators is hormones and growth factor activators, including 4-methyl catechol derivatives like 4-methylcatechol acetic acid (MCBA) that stimulate the synthesis and/or activation of growth factors such as NGF and BDNF, which also activate PKC as well as convergent pathways responsible for synaptogenesis and/or neuritic branching.

Further example PKC activators include polyunsaturated fatty acids (“PUFAs”). These compounds are essential components of the nervous system and have numerous health benefits. In general, PUFAs increase membrane fluidity, rapidly oxidize to highly bioactive products, produce a variety of inflammatory and hormonal effects, and are rapidly degraded and metabolized. The inflammatory effects and rapid metabolism is likely the result of their active carbon-carbon double bonds. These compounds may be potent activators of PKC, most likely by binding the PS site.

In one embodiment, the PUFA is chosen from linoleic acid (shown below).

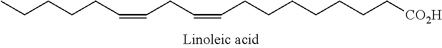 [SMILES: CCCCC/C=C\C/C=C\CCCCCCCC(=O)O]

Another class of PKC activators is PUFA and MUFA derivatives, and cyclopropanated derivatives in particular. Certain cyclopropanated PUFAs, such as DCPLA (i.e., linoleic acid with cyclopropane at both double bonds), may be able to selectively activate PKC-ε. See Journal of Biological Chemistry, 2009, 284(50): 34514-34521; see also U.S. Patent Application Publication No. 2010/0022645 A1. Like their parent molecules, PUFA derivatives are thought to activate PKC by binding to the PS site.

Cyclopropanated fatty acids exhibit low toxicity and are readily imported into the brain where they exhibit a long half-life (t_{1/2}). Conversion of the double bonds into cyclopropane rings prevents oxidation and metabolism to inflammatory byproducts and creates a more rigid U-shaped 3D structure that may result in greater PKC activation. Moreover, this U-shape may result in greater isoform specificity. For example, cyclopropanated fatty acids may exhibit potent and selective activation of PKC-ε.

The Simmons-Smith cyclopropanation reaction is an efficient way of converting double bonds to cyclopropane groups. This reaction, acting through a carbenoid intermediate, preserves the cis-stereochemistry of the parent molecule. Thus, the PKC-activating properties are increased while metabolism into other molecules like bioreactive eicosanoids, thromboxanes, or prostaglandins is prevented.

One class of PKC-activating fatty acids is Omega-3 PUFA derivatives. In one embodiment, the Omega-3 PUFA derivatives are chosen from cyclopropanated docosahexaenoic acid, cyclopropanated eicosapentaenoic acid, cyclopropanated rumelenic acid, cyclopropanated parinaric acid, and cyclopropanated linolenic acid (CP3 form shown below).

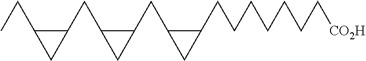 [SMILES: CCC1CC1CC1CC1CC1CC1CCCCCCCC(=O)O]

Another class of PKC-activating fatty acids is Omega-6 PUFA derivatives. In one embodiment, the Omega-6 PUFA derivatives are chosen from cyclopropanated linoleic acid (“DCPLA,” CP2 form shown below),

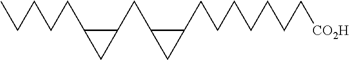 [SMILES: CCCCCC1CC1CC1CC1CCCCCCCC(=O)O]

cyclopropanated arachidonic acid, cyclopropanated eicosadienoic acid, cyclopropanated dihomo-gamma-linolenic acid, cyclopropanated docosadienoic acid, cyclopropanated adrenic acid, cyclopropanated calendic acid, cyclopropanated docosapentaenoic acid, cyclopropanated jacaric acid, cyclopropanated pinolenic acid, cyclopropanated podocarpic acid, cyclopropanated tetracosatetraenoic acid, and cyclopropanated tetracosapentaenoic acid.

Vernolic acid is a naturally occurring compound. However, it is an epoxyl derivative of linoleic acid and therefore, as used herein, is considered an Omega-6 PUFA derivative. In addition to vernolic acid, cyclopropanated vernolic acid (shown below) is an Omega-6 PUFA derivative.

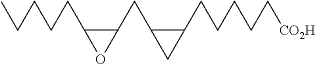 [SMILES: CCCCCC1OC1CC1CC1CCCCCC(=O)O]

Another class of PKC-activating fatty acids is Omega-9 PUFA derivatives. In one embodiment, the Omega-9 PUFA derivatives are chosen from cyclopropanated eicosenoic acid, cyclopropanated mead acid, cyclopropanated erucic acid, and cyclopropanated nervonic acid.

Yet another class of PKC-activating fatty acids is monounsaturated fatty acid (“MUFA”) derivatives. In one embodiment, the MUFA derivatives are chosen from cyclopropanated oleic acid (shown below),

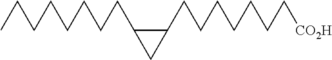 [SMILES: CCCCCCCCC1CC1CCCCCCCC(=O)O]

and cyclopropanated elaidic acid (shown below).

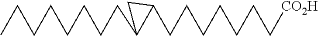 [SMILES: CCCCCCCCC1CC1CCCCCCCC(=O)O]

PKC-activating MUFA derivatives include epoxylated compounds such as trans-9,10-epoxystearic acid (shown below).

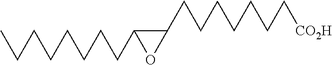 [SMILES: CCCCCCCCC1OC1CCCCCCCC(=O)O]

Another class of PKC-activating fatty acids is Omega-5 and Omega-7 PUFA derivatives. In one embodiment, the Omega-5 and Omega-7 PUFA derivatives are chosen from cyclopropanated rumenic acid, cyclopropanated alpha-elostearic acid, cyclopropanated catalpic acid, and cyclopropanated punicic acid.

Another class of PKC activators is fatty acid alcohols and derivatives thereof, such as cyclopropanated PUFA and MUFA fatty alcohols. It is thought that these alcohols activate PKC by binding to the PS site. These alcohols can be derived from different classes of fatty acids.

In one embodiment, the PKC-activating fatty alcohols are derived from Omega-3 PUFAs, Omega-6 PUFAs, Omega-9 PUFAs, and MUFAs, especially the fatty acids noted above. In one embodiment, the fatty alcohol is chosen from cyclopropanated linolenyl alcohol (CP3 form shown below),

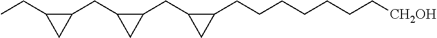 [SMILES: CCC1CC1CC1CC1CC1CC1CCCCCCCCO]

cyclopropanated linoleyl alcohol (CP2 form shown below),

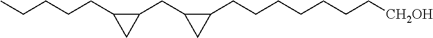 [SMILES: CCCCCC1CC1CC1CC1CCCCCCCCO]

cyclopropanated elaidic alcohol (shown below),

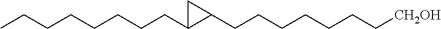 [SMILES: CCCCCCCCC1CC1CCCCCCCCO]

cyclopropanated DCPLA alcohol, and cyclopropanated oleyl alcohol.

Another class of PKC activators is fatty acid esters and derivatives thereof, such as cyclopropanated PUFA and MUFA fatty esters. In one embodiment, the cyclopropanated fatty esters are derived from Omega-3 PUFAs, Omega-6 PUFAs, Omega-9 PUFAs, MUFAs, Omega-5 PUFAs, and Omega-7 PUFAs. These compounds are thought to activate PKC through binding on the PS site. One advantage of such esters is that they are generally considered to be more stable that their free acid counterparts.

In one embodiment, the PKC-activating fatty acid esters derived from Omega-3 PUFAs are chosen from cyclopropanated eicosapentaenoic acid methyl ester (CP5 form shown below)

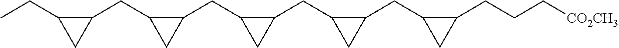 [SMILES: CCC1CC1CC1CC1CC1CC1CC1CC1CC1CC1CCCC(=O)OC]

and cyclopropanated linolenic acid methyl ester (CP3 form shown below).

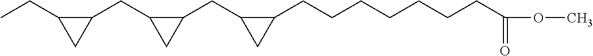 [SMILES: CCC1CC1CC1CC1CC1CC1CCCCCCCC(=O)OC]

In another embodiment, the Omega-3 PUFA esters are chosen from esters of DHA-CP6 and aliphatic and aromatic alcohols. In one embodiment, the ester is cyclopropanated docosahexaenoic acid methyl ester (CP6 form shown below).

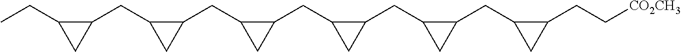 [SMILES: CCC1CC1CC1CC1CC1CC1CC1CC1CC1CC1CC1CC1CCC(=O)OC]

DHA-CP6, in fact, has been shown to be effective at a concentration of 10 nM. See, e.g., U.S. Patent Application Publication No. 2010/0022645.

In one embodiment, PKC-activating fatty esters derived from Omega-6 PUFAs are chosen from cyclopropanated arachidonic acid methyl ester (CP4 form shown below),

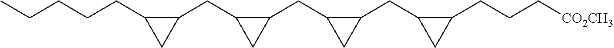 [SMILES: CCCCCC1CC1CC1CC1CC1CC1CC1CC1CCCC(=O)OC]

cyclopropanated vernolic acid methyl ester (CP1 form shown below), and

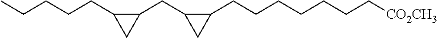 [SMILES: CCCCCC1CC1CC1CC1CCCCCCCC(=O)OC]

vernolic acid methyl ester (shown below).

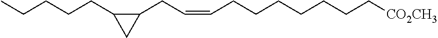 [SMILES: CCCCCC1CC1C/C=C\CCCCCCCC(=O)OC]

One particularly interesting class of esters are derivatives of DCPLA (CP6-linoleic acid). See, e.g., U.S. Provisional Patent Application No. 61/559,117 and applications claiming priority thereof. In one embodiment, the ester of DCPLA is an alkyl ester. The alkyl group of the DCPLA alkyl esters may be linear, branched, and/or cyclic. The alkyl groups may be saturated or unsaturated. When the alkyl group is an unsaturated cyclic alkyl group, the cyclic alkyl group may be aromatic. The alkyl group, in one embodiment, may be chosen from methyl, ethyl, propyl (e.g., isopropyl), and butyl (e.g., tert-butyl) esters. DCPLA in the methyl ester form (“DCPLA-ME”) is shown below.

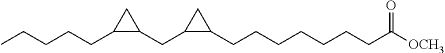 [SMILES: CCCCCC1CC1CC1CC1CCCCCCCC(=O)OC]

In another embodiment, the esters of DCPLA are derived from a benzyl alcohol (unsubstituted benzyl alcohol ester shown below). In yet another embodiment, the esters of DCPLA are derived from aromatic alcohols such as phenols used as antioxidants and natural phenols with pro-learning ability. Some specific examples include estradiol, butylated hydroxytoluene, resveratrol, polyhydroxylated aromatic compounds, and curcumin.

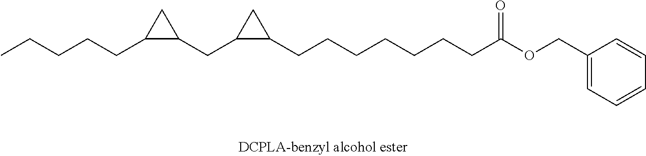 [SMILES: CCCCCC1CC1CC1CC1CCCCCCCC(=O)OCc1ccccc1]

Another class of PKC activators is fatty esters derived from cyclopropanated MUFAs. In one embodiment, the cyclopropanated MUFA ester is chosen from cyclopropanated elaidic acid methyl ester (shown below),

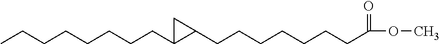 [SMILES: CCCCCCCCC1CC1CCCCCCCC(=O)OC]

and cyclopropanated oleic acid methyl ester (shown below).

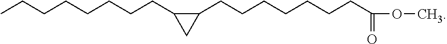 [SMILES: CCCCCCCCC1CC1CCCCCCCC(=O)OC]

Another class of PKC activators is sulfates and phosphates derived from PUFAs, MUFAs, and their derivatives. In one embodiment, the sulfate is chosen from DCPLA sulfate and DHA sulfate (CP6 form shown below).

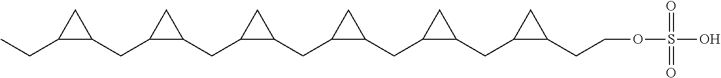 [SMILES: CCC1CC1CC1CC1CC1CC1CC1CC1CC1CC1CC1CC1CCOS(=O)(=O)O]

In one embodiment, the phosphate is chosen from DCPLA phosphate and DHA phosphate (CP6 form shown below).

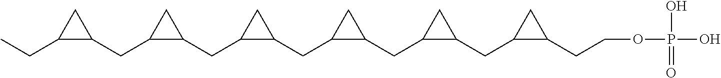 [SMILES: CCC1CC1CC1CC1CC1CC1CC1CC1CC1CC1CC1CC1CCOP(=O)(O)O]

In one embodiment the PKC activator is a macrocyclic lactone, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, or fatty acid esters.

As illustrated above, bryostatin-1 reduces skin aging by, e.g., promoting the expression of collagen and structural proteins in dermal fibroblast cells. Prolonged treatment of dermal fibroblast cells with bryostatin-1 was observed to reduce the expression of caspase-8, a cysteine-aspartic protease with known apoptotic activity. Since healthy, viable fibroblast cells are necessary for skin health dermal compositions of bryostatin-1 or an analog of bryostatin-1 such as picolog are candidate therapeutic agents for reducing the appearance of aging.

FIG. 5A shows microscopic images of dermal fibroblasts that were cultured in serum free medium containing bryostatin-1 (treated group) or in the absence of bryostatin-1 (control group). For the bryostatin-1 treated group, there is no significant loss of dermal fibroblast cells after 15 days in serum free culture. While the percent loss of dermal fibroblast cells in the treated group increased after 35 days in serum free culture, cells in the treated group showed greater structural integrity and a higher number of live, viable cells than cells in the control group.

Gel electrophoresis of the lysate from bryostatin-1 treated cells and control fibroblast cells show reduced expression levels of caspase-8 in the lysate of cells from the bryostatin-1 treated group (FIG. 5B). In fact, the greatest decrease in caspase-8 expression levels were seen after 3 days of treatment with Bryostatin-1 (FIG. 5C). These results support bryostatin's role in inhibiting caspase-8 expression. Because loss of dermal fibroblast cells accompanies skin aging, the present inventors propose compositions comprising bryostatin-1 as therapeutic agents for reducing the appearance of aging.

In addition to its role in combating aging, bryostain-1 and picolog can decrease the formation of scar tissue following surgical intervention or injury to skin. In one embodiment, the disclosure provides a method for decreasing the formation of scar tissue in a human following injury by applying a topical composition of a PKC activator or an analog of a PKC activator at the site of injury.

According to this embodiment, the scar tissue forms during the healing of a cut, or some surface injury to skin. According to the method a sterile bandage is placed over the topical composition at the site of skin injury to protect the injured site from infection and from further injury. The sterile bandage typically is attached to a surface of normal skin in the vicinity of the injury, or surrounding the site of injury. Within the context of this disclosure, the term “sterilized” refers to the state of being substantially free of living microorganisms, or being subject to a process in order to be substantially free of living microorganisms.

According to another embodiment, the disclosed method decreases the formation of scar tissue following a surgical procedure. Post-operative scar tissue formation and skin adhesions are major problems following abdominal, neurological, vascular or other types of surgery. In some instances, the formation of scar tissue can prevent healing, especially if the scar tissue abuts blood vessels around the site of surgery and causes a narrowing of such blood vessels. The inventive method prevents or decreases the formation of scar tissue by using a topical composition of a PKC activator or an analog of a PKC activator as the therapeutic agent.

In one aspect of the method, the PKC activator or an analog of a PKC activator is directly applied in a therapeutically effective amount at the site of a surgical procedure.

According to another embodiment, the PKC activator or an analog of a PKC activator is placed on a sterile mesh prior to its application at the site of a surgical procedure. The sterile mesh used is adapted for placement at the site of surgery. Thus the mesh used can be bent or cut to conform the mesh to a shape and size appropriate for placement at the surgical site. The mesh can cover the site of surgical intervention or can entirely wrap around some human tissue at the site of a surgical procedure.

In one embodiment, the mesh is attached to the surgical site by surgical sutures or surgical staples. The mesh can be made of biodegradable material or a non-immunogenic material that permits permanent implantation of the mesh at the site of surgical intervention. Exemplary meshes are SURGICEL™ manufactured by Johnson & Johnson, an absorbable hemostat gauze-like sheet or a Vicryl polymer mesh product.

The disclosed method also decreases the formation of scar tissue at a site of anastomosis or at a site where sutures or staples are used to close a cut or surgical insertion. Accordingly, in one embodiment, sutures or staples coated or impregnated with a PKC activator or an analog of a PKC activator are used to decrease the formation of scar tissue. Such sutures or staples are especially useful to plastic surgeons for reconstructive procedures where minimization of any form of scarring or visual aspects of surgical intervention are highly desired.

According to another aspect of the method, the PKC activator or an analog of a PKC activator is embedded into the mesh or a surgical suture. Alternatively, the PKC activator or an analog of a PKC activator is coated onto a surface of a mesh, a surgical staple, or a surgical suture.

To decrease the formation of surface scar tissue, the method provides a topical composition of a PKC activator or an analog of a PKC activator. Typically, the topical composition comprises a single PKC activator that is formulated using a dermatologically acceptable carrier. In certain embodiments, however, the topical composition can contain two different PKC activators, or a combination of a PKC activator and a second therapeutic agent.

According to the disclosed method, the topical composition is applied over an extended period of time, so as to decrease or prevent the formation of scar tissue as the skin heals. Thus, the topical composition can be applied for a period of time from about 1 day to about 90 days, about 3 days to about 60 days, about 5 days to about 45 days, about 7 days to about 30 days.

The topical composition used in the disclosed method is applied once a day, or multiple times during a 24 hour period. In one aspect, the topical composition is applied every 12 hours.

According to another aspect, the topical composition is applied every 8 hours, every 6 hours, every 4 hours, every 2 hours or every hour.

In one embodiment, the PKC activator in a topical composition of the disclosed method is bryostatin. According to another embodiment, the PKC activator is a bryolog. Illustrative of the category “bryostatin” are bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

In one embodiment the topical composition comprises picolog, which is a synthetic analog of bryostatin-1.

If a second therapeutic agent is present in the topical composition, such an agent can be an antibiotic, an anti-inflammatory agent, an agent that promotes angiogenesis, or compounds that inhibit excess biosynthesis of collagen. Exemplary antibiotics for use in the topical compositions of the disclosed method are compounds belonging to the penicillin family, cephalosporins, rifamycins, sulfonamides, quinolones, oxazolidones, tetracyclines and cyclic lipopeptides. The manufacture of formulations containing two or more agents is carried out using established principles of pharmaceutical compounding.

The methods for decreasing the formation of scar tissue are effective on scars that are one or more weeks old, including scars that have been present for at least 1 month, at least 2 months, at least 3 months, at least 4 months, at least 5 months, at least 6 months, at least 7 months, at least 8 months, at least 9 months, at least 10 months, at least 11 months, or at least 12 months.

In one embodiment of the method, the composition comprising a PKC activator or an analog of the PKC activator is applied to a scar at the time of provisional scar matrix formation to effect subsequent matrix dissolution and maturation.

In one embodiment, the topical composition is applied at the time of injury, or within a day or two thereafter. In another embodiment, the wound is treated within 1-4 weeks of wound occurrence. In several embodiments, the method comprises applying the topical composition to a scar that is weeks, months or years old to improve the appearance of the scar.

According to another embodiment, the treatment of a scar is commenced immediately following wound closure, for example, following surgical suturing.

The composition of a PKC activator or an analog of a PKC activator when used according to the disclosed method decreases scar tissue formation by at least 25% to at least 95% compared to an untreated wound. In one embodiment, the percent decrease in scar tissue is about 25%, about 30%, about 35%, about 40%, about 45%, about 50%, about 55%, about 60%, about 65%, about 70%, about 75%, about 80%, about 85%, or about 90%, compared to an untreated wound.

The normal process of wound healing is slowed or becomes ineffective in diabetic patients. While the underlying pathophysiology for wound healing in diabetics is complex, i a portion of diabetics will develop foot ulcers during their lifetime. New methods for treating wounds and therapies for promoting wound healing in diabetics are needed and the present disclosure serves this need.

In one aspect, the application provides a method for promoting wound-healing in a diabetic subject by providing a composition of a PKC activator or an analog of a PKC activator. Also disclosed is a method for treating a wound and a method for healing wounds in a diabetic subject.

According to the disclosed method, wound-healing is promoted by the activation of dermal fibroblast cells. Activation of dermal fibroblasts results in the synthesis of collagen, structural proteins and proteoglycans that are necessary components for cellular morphology and for formation of new tissue. Activation of dermal fibroblasts also promotes cell proliferation to the site of wound and thus aids in the repair and healing of wound. In one embodiment, a PKC activator or an analog of a PKC activator is provided for promoting wound healing in a diabetic subject.

The PKC activator or an analog of a PKC activator can be administered orally, intravenously, bucally, or formulated as a topical gel or cream for use in the disclosed method.

In one embodiment, the method promotes the healing of ulcers, especially foot ulcers commonly associated with diabetic foot syndrome. According to the disclosed method, the PKC activator or an analog of the PKC activator may be used alone for healing wounds or in combination with therapeutic agents that exert a control on sugar metabolism as well as therapeutic agents that restore balance between the accumulation of collagenous and noncollagenous extracellular matrix components necessary to promote wound healing.

Wound healing is determined by MMPs and tissue inhibitors of metalloproteinases (TIMPs). MMPs are primarily responsible for initial debridement of a wound as well as angiogenesis while TIMPs inhibit MMPs and promote the formation of new tissue by virtue of their cell growth promoting activity.

As described above, the Bryostatin class of compounds are inhibitors of several MMPs. For example, bryostatin-1 is an inhibitor of MMP-1, MMP-3, MMP-9, MMP-10, and MMP-11. According to one embodiment, pharmaceutical compositions comprising a therapeutically effective amount of bryostatin-1 in a suitable pharmaceutical carrier is a candidate therapeutic agent for healing wounds in a diabetic subject in need of such treatment.

In one embodiment, the composition for healing a wound or promoting wound healing contains bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

According to another embodiment the composition contains a therapeutically effective amount of synthetic analog of bryostatin-1, picolog, in a suitable pharmaceutical carrier.

For certain types of wounds, for example, wounds that cause loss of significant amounts of skin tissue, it may be necessary to graft skin obtained from a donor at the site of injury. According to the application, the clinical acceptance of a skin graft is improved by contacting donor skin with a composition comprising a PKC activator prior to grafting.

In one aspect, of the inventive method acceptance of a skin graft is improved by contacting donor skin with a cocktail containing fibroblast growth factors, hormones and one or more PKC activators or an analog of a PKC activator prior to surgical grafting.

According to another embodiment, donor skin is contacted with a cocktail containing fibroblast growth factors, hormones and one or more PKC activators or an analog of a PKC activator following surgical grafting.

According to another embodiment, contact of donor skin with a cocktail containing fibroblast growth factors, hormones and one or more PKC activators or an analog of a PKC activator is established before surgical grafting, with further application of the cocktail to the grafted donor skin following surgical placement of the graft at the site of a wound.

In one embodiment, the donor skin is contacted with a cocktail containing fibroblast growth factors, hormones, vitamins, amino acids, hyaluronic acid (HA), and one or more PKC activators or an analog of a PKC activator before surgical grafting.

Contact of the donor skin with one or more PKC activators or an analog of a PKC activator is for a period of at least 2 hours, at least 4 hours, at least 6 hours, at least 8 hours, at least 10 hours, at least 12 hours, at least 14 hours, at least 16 hours, at least 18 hours, at least 20 hours, at least 22 hours, or at least 24 hours, before the donor skin is surgically grafted.

According to another embodiment, donor skin is contacted with one or more PKC activators or an analog of a PKC activator for at least 2 days, at least 3 days, at least 4 days, at least 5 days, at least 6 days, at least 7 days, before surgical grafting at the site of wound.

In one embodiment, donor skin is soaked in a physiologically acceptable solution of a PKC activator or an analog of a PKC activator prior to grafting. Alternatively, a physiologically acceptable solution of a PKC activator or an analog of a PKC activator is applied or sprayed onto the donor skin prior to grafting.

The amount of a PKC activator or an analog of a PKC activator applied to a donor skin is from about 0.001 nmoles to about 10 μmoles, from about 0.001 nmoles to about 9 μmoles, from about 0.001 nmoles to about 8 μmoles, from about 0.001 nmoles to about 7 μmoles, from about 0.001 nmoles to about 6 μmoles, from about 0.001 nmoles to about 5 μmoles, from about 0.001 nmoles to about 4 μmoles, from about 0.001 nmoles to about 3 μmoles, from about 0.001 nmoles to about 2 μmoles, or from about 0.001 nmoles to about 1 μmoles.

For certain embodiments, the amount of a PKC activator or an analog of a PKC activator applied to a donor skin is from about 0.001 nmoles to about 800 nmoles, from about 0.001 nmoles to about 700 nmoles, from about 0.001 nmoles to about 600 nmoles, from about 0.001 nmoles to about 500 nmoles, from about 0.001 nmoles to about 400 nmoles, from about 0.001 nmoles to about 300 nmoles, from about 0.001 nmoles to about 200 nmoles, or from about 0.001 nmoles to about 100 nmoles.

In one embodiment the amount of a PKC activator or an analog of a PKC activator applied to a donor skin is from about 0.001 nmoles to about 95 nmoles, from about 0.001 nmoles to about 90 nmoles, from about 0.001 nmoles to about 80 nmoles, from about 0.001 nmoles to about 70 nmoles, from about 0.001 nmoles to about 60 nmoles, from about 0.001 nmoles to about 50 nmoles, from about 0.001 nmoles to about 45 nmoles, from about 0.001 nmoles to about 40 nmoles, from about 0.001 nmoles to about 35 nmoles, from about 0.001 nmoles to about 30 nmoles, from about 0.001 nmoles to about 25 nmoles, from about 0.001 nmoles to about 20 nmoles, from about 0.001 nmoles to about 15 nmoles, from about 0.001 nmoles to about 10 nmoles, or from about 0.001 nmoles to about 5 nmoles.

In one aspect, the amount of a PKC activator or an analog of a PKC activator applied to a donor skin is from about 0.001 nmoles to about 1 nmoles, from about 0.001 nmoles to about 0.9 nmoles, from about 0.001 nmoles to about 0.8 nmoles, from about 0.001 nmoles to about 0.7 nmoles, from about 0.001 nmoles to about 0.6 nmoles, from about 0.001 nmoles to about 0.5 nmoles, from about 0.001 nmoles to about 0.4 nmoles, from about 0.001 nmoles to about 0.3 nmoles, from about 0.001 nmoles to about 0.2 nmoles, from about 0.001 nmoles to about 0.1 nmoles, or from about 0.001 nmoles to about 0.01 nmoles.

Clinical protocols for assessing the efficacy of the disclosed method for decreasing the formation of scar tissue are well known in the medical field, but include without limitation observations related to a decrease in the roughness of a scar, decrease in the elevation of a scar, decrease in the color of a scar as well as decrease in the size of a scar.

Exemplary fibroblast growth factors, hormones and biological agents used to prepare a cocktail for contacting a donor skin according to the described method include without limitation bovine pituitary extract, insulin, human epithelial growth factor, transferrin, epinephrine, and hydrocortisone.

Donor skin suitable for grafting according to a method of the application can be obtained from a different site on a subject receiving the skin graft or from a donor unrelated to the subject. Thus, the method encompasses skin grafts that are autologous, isogeneic, allogeneic, and xenogeneic graft tissue. The graft can be a split-thickness skin graft or a full-thickness skin graft. To prevent host-graft rejection, immunosuppressive agents are administered following graft surgery. However, the type of immunosuppressive agent used, the duration of administration of the immunosuppressive agent, the route of administration and dose administered will depend on several factors, including the patient's health, age, and administration of an immunosuppressive agent, therefore, is at the discretion of the prescribing physician.

Skin wounds that can receive a graft according to the method of the invention include without limitation wounds associated with late stage diabetic foot ulcers, necrotizing faciitis, or wounds from physical trauma such as those arising from an amputation, an accident or a burn.

The one or more PKC activator or a combination of PKC activators may be administered to a patient/subject in need thereof by conventional methods such as oral, parenteral, transmucosal, intranasal, inhalation, or transdermal administration. Parenteral administration includes intravenous, intra-arteriolar, intramuscular, intradermal, subcutaneous, intraperitoneal, intraventricular, intrathecal, ICV, intracisternal injections or infusions and intracranial administration.

The present disclosure relates to compositions comprising one or more protein kinase C activator or combinations thereof and a carrier. The present disclosure further relates to a composition of at least one protein kinase C activator and a carrier, and a composition of at least one combination of a PKC activator an analog of a PKC activator and a carrier, wherein the two compositions are administered together to a patient in need thereof. In one embodiment, the composition of at least one protein kinase C activator may be administered before or after the administration of the composition of the combination to a patient in need thereof.

The formulations of the compositions described herein may be prepared by any suitable method known in the art. In general, such preparatory methods include bringing at least one of active ingredients into association with a carrier. If necessary or desirable, the resultant product can be shaped or packaged into a desired single- or multi-dose unit.

Although the descriptions of compositions provided herein are principally directed to compositions suitable for ethical administration to humans, it will be understood by a skilled artisan that such compositions are generally suitable for administration to animals of all sorts. Modification of pharmaceutical compositions suitable for administration to humans or to render the compositions suitable for administration to various animals is well understood, and the ordinarily skilled veterinary pharmacologist can design and perform such modification with merely ordinary, if any, experimentation. Subjects to which administration of the compositions of the disclosure is contemplated include, but are not limited to, humans and other primates, and other mammals.

As discussed herein, carriers include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the compositions of the disclosure are generally known in the art and may be described, for example, in Remington's Pharmaceutical Sciences, Genaro, ed., Mack Publishing Co., Easton, Pa., 1985, and Remington's Pharmaceutical Sciences, 20^{th} Ed., Mack Publishing Co. 2000, both incorporated by reference herein.

In one embodiment, the carrier is an aqueous or hydrophilic carrier. In a further embodiment, the carrier can be water, saline, or dimethylsulfoxide. In another embodiment, the carrier is a hydrophobic carrier. Hydrophobic carriers include inclusion complexes, dispersions (such as micelles, microemulsions, and emulsions), and liposomes. Exemplary hydrophobic carriers include inclusion complexes, micelles, and liposomes. See, e.g., Remington's: The Science and Practice of Pharmacy 20th ed., ed. Gennaro, Lippincott: Philadelphia, Pa. 2003, incorporated by reference herein. In addition, other compounds may be included either in the hydrophobic carrier or the solution, e.g., to stabilize the formulation.

The compositions disclosed herein may be administrated to a patient in need thereof by any suitable route including oral, parenteral, transmucosal, intranasal, inhalation, or transdermal routes. Parenteral routes include intravenous, intra-arteriolar, intramuscular, intradermal, subcutaneous, intraperitoneal, intraventricular, intrathecal, and intracranial administration. A suitable route of administration may be chosen to permit crossing the blood-brain barrier. See e.g., J. Lipid Res. (2001) vol. 42, pp. 678-685, incorporated by reference herein.

In one embodiment, the compositions described herein may be formulated in oral dosage forms. For oral administration, the composition may take the form of a tablet or capsule prepared by conventional means with, for example, carriers such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods generally known in the art.

In another embodiment, the compositions herein are formulated into a liquid preparation. Such preparations may take the form of, for example, solutions, syrups or suspensions, or they may be presented as a dry product for constitution with water or other suitable vehicle before use. Such liquid preparations may be prepared by conventional means with, for examples, pharmaceutically acceptable carriers such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl p-hydroxybenzoates, or sorbic acid). The preparations may also comprise buffer salts, flavoring, coloring, and sweetening agents as appropriate. In one embodiment, the liquid preparation is for oral administration.

In another embodiment of the present disclosure, the compositions herein may be formulated for parenteral administration such as bolus injection or continuous infusion. Formulations for injection may be presented in unit dosage form, e.g., in ampoules, or in multi-dose containers, with an added preservative. The compositions may take such forms as suspensions, solutions, dispersions, or emulsions in oily or aqueous vehicles, and may contain formulatory agents such as suspending, stabilizing, and/or dispersing agents.

In another embodiment, the compositions herein may be formulated as depot preparations. Such formulations may be administered by implantation (for example, subcutaneously or intramuscularly) or by intramuscular injection. For example, the compositions may be formulated with a suitable polymeric or hydrophobic material (for example, as an emulsion in an acceptable oil) or ion exchange resin, or as a sparingly soluble derivative, for example, as a sparingly soluble salt.

In another embodiment, at least one PKC activator or combination thereof is delivered in a vesicle, such as a micelle, liposome, or an artificial low-density lipoprotein (LDL) particle. See, e.g., U.S. Pat. No. 7,682,627.

In a further embodiment, the doses for administration to a patient in need thereof may suitably be prepared so as to deliver from about 0.001 mg to about 1.0 g, such as from about 1.0 mg to about 0.9 g, from about 0.5 mg to about 0.8 g, from about 0.2 mg to about 0.5 g, from about 0.1 mg to about 0.2 g, or from about 0.08 mg to about 0.1 g.

For some embodiments, doses for administration to a patient are prepared to deliver about 0.001 mg to about 0.08 mg, from about 0.001 mg to about 0.06 mg, from about 0.001 mg to about 0.04 mg, from about 0.001 mg to about 0.02 mg, from about 0.001 mg to about 0.008 mg, from about 0.001 mg to about 0.006 mg, from about 0.001 mg to about 0.004 mg, about 0.003 mg, about 0.002 mg, or 0.0015 mg.

In one embodiment, at least one PKC activator or combination thereof may be present in the composition in an amount ranging from about 0.01% to about 100%, from about 0.1% to about 90%, from about 0.1% to about 60%, from about 0.1% to about 30% by weight, or from about 1% to about 10% by weight of the final formulation. In another embodiment, at least one PKC activator or combination thereof may be present in the composition in an amount ranging from about 0.01% to about 100%, from about 0.1% to about 95%, from about 1% to about 90%, from about 5% to about 85%, from about 10% to about 80%, and from about 25% to about 75%.

The kits may comprise devices for storage and/or administration. For example, the kits may comprise syringe(s), needle(s), needle-less injection device(s), sterile pad(s), swab(s), vial(s), ampoule(s), cartridge(s), bottle(s), and the like. The storage and/or administration devices may be graduated to allow, for example, measuring volumes. In one embodiment, the kit comprises at least one PKC activator in a container separate from other components in the system. In another embodiment, the kit comprises a means to combine at least one PKC activator and at least one combination separately. In yet another embodiment, the kit comprises a container comprising at least one PKC activator and a combination thereof.

The kits may also comprise one or more anesthetics, such as local anesthetics. In one embodiment, the anesthetics are in a ready-to-use formulation, for example an injectable formulation (optionally in one or more pre-loaded syringes), or a formulation that may be applied topically. Topical formulations of anesthetics may be in the form of an anesthetic applied to a pad, swab, towelette, disposable napkin, cloth, patch, bandage, gauze, cotton ball, Q-Tip™, ointment, cream, gel, paste, liquid, or any other topically applied formulation. Anesthetics for use with the present disclosure may include, but are not limited to lidocaine, marcaine, cocaine, and xylocaine.

The kits may also contain instructions relating to the use of at least one PKC activator or a combination thereof. In another embodiment, the kit may contain instructions relating to procedures for mixing, diluting, or preparing formulations of at least one PKC activator or a combination thereof. The instructions may also contain directions for properly diluting a formulation of at least one PKC activator or a combination thereof in order to obtain a desired pH or range of pHs and/or a desired specific activity and/or protein concentration after mixing but prior to administration. The instructions may also contain dosing information. The instructions may also contain material directed to methods for selecting subjects for treatment with at least one PKC activator or a combination thereof.

The PKC activator can be formulated, alone in suitable dosage unit formulations containing conventional non-toxic pharmaceutically acceptable carriers, adjuvants and vehicles appropriate for each route of administration. Pharmaceutical compositions may further comprise other therapeutically active compounds which are approved for the treatment of neurodegenerative diseases or to reduce the risk of developing a neurodegenerative disorder.

Appropriate dosages of the PKC activator will generally be about 0.001 to 100 μg/m²/week which can be administered in single or multiple doses. For example, the dosage level will be about 0.01 to about 25 μg/m²/week; about 1 to about 20 μg/m²/week, about 5 to about 20 μg/m²/week, or about 10 to about 20 μg/m²/week. A suitable dosage may be about 5 μg/m²/week, about 10 μg/m²/week, about 15 μg/m²/week, or about 20 μg/m²/week.

For oral administration, the compositions are preferably provided in the form of tablets containing about 1 to 1000 micrograms of the active ingredient, particularly about 1, 5, 10, 15, 20, 25, 50, 75, 100, 150, 200, 250, 300, 400, 500, 600, 750, 800, 900, and 1000 micrograms of an active ingredient such as a PKC activator.

The pharmaceutical compositions according to the invention can be administered more than once a week, for example, using a regimen that comprises administering the composition 2, 3, 4, or 5 times a week. For certain neurodegenerative conditions, the pharmaceutical composition is administered daily, for example, once per day, twice per day, or at regular intervals of time such as weekly or every other week, two weeks, three weeks or four weeks.

It will be understood, however, that the specific dose and frequency of dosage for any particular patient may be varied and will depend upon a variety of factors including the activity of the compound formulated, the metabolic stability and length of action of that compound, the age, body weight, general health, sex, diet, mode and time of administration, rate of excretion, drug combinations used, and the severity of the particular neurodegenerative condition.

### Examples

### I. Materials and Methods

Bryostatin1 (Cat#2383) was purchased from Tocris Bioscience (Minneapolis, Minn.). The Erk inhibitor PD98059 (Cat# P215) was purchased from Sigma-Aldrich (St. Louis, Mo.). P44/42 (Erk1/2) rabbit monoclonal antibody (Cat#4695) and phosphoP44/42 MAPK (Erk1/2) (Thr202/Tyr204) rabbit polyclonal antibody (Cat#9101) were purchased from Cell Signaling Technology (Danvers, Mass.). Human/mouse caspase-8 antibody (Cat# AF1650) was purchased from R&D Systems (Minneapolis, Minn.). β-actin (AC-15) antibody (Cat#sc-69879) was purchased from Santa Cruz Biotechnology, Inc. (Santa Cruz, Calif.). The design and synthesis of synthetic analog of bryostatin1, picolog, were conducted according to published methods (Wender et al, 2002).

### II. Cell Culture

A. Primary Dermal Cell Lines:

Skin fibroblast samples AG09555 (isolated and banked from a 53-year-old healthy female) and AG04560 (isolated and banked from a 59-year-old healthy male; Aging Cell Culture Repository of the National Institute on Aging) were obtained from the Coriell Institute for Medical Research (Camden, N.J.). Fresh fibroblasts were isolated from a skin punch biopsy (2-3 mm, upper arm) from a 45-year-old healthy female subject (0078F45). The skin biopsy was conducted by qualified personnel under the supervision of Dr. Shirley Neitch with IRB approval of Marshall University (Huntington, W. Va.). All subjects signed informed consent forms under Marshal University rules and regulations. The method for isolating fibroblasts from skin biopsies was similar to the method previously described (Zhao et al, 2002; Khan and Alkon, 2006).

B. Dermal Fibroblast Culture and Treatment:

Primary skin fibroblasts isolated from skin biopsies were maintained in Dulbecco's Modified Eagle Medium (DMEM) with low glucose (Invitrogen, Grand Island, N.Y.) supplemented with 10% FBS in 6-well culture dishes (37° C., 5% CO₂, 90% humidity) until the cells reached 90-100% confluence. Cells were cultured in completely serum-free medium to simulate stress conditions. After culture in serum-free media for 24 hours, cells were treated with vehicle, 0.3 nM bryostatin1, or 5 nM picolog. We previously found that sub-nanomolar concentrations of bryostatin1 are effective for cellular activation (Khan et al, 2009) and we used same 0.3 nM dose for all experiments. Nanomolar concentrations of picolog were previously found to be effective for cellular activation (Khan et al, 2009). The condition of the cultured skin fibroblasts was monitored with an inverted cell culture microscope (Westover Digital AMID Model 2000, Westover Scientific, Bothell, Wash.), controlled by a computer and images were captured with image acquisition software (Micron 2.0.0, Westover Scientific).

C. Epidermal Keratinocyte Culture:

The Clonetics™ keratinocyte system containing normal adult human epidermal keratinocytes (NHEK) was used as source of epidermal keratinocytes (Lonza, Walkersville, Md.). Human adult epidermal keratinocyte cells were cultured in keratinocyte basal medium supplemented with appropriate growth factors (Clonetics KGM-Gold™ BulletKit™ [cat#00192060] contains one 500 ml bottle of keratinocyte basal medium-gold supplemented with bovine pituitary extract (BPE), 2 ml; hEGF, 0.5 ml; insulin, 0.5 ml; hydrocortisone, 0.5 ml; transferrin, 0.5 ml; epinephrine, 0.25 ml; GA-1000, 0.5 ml) in T25 culture flasks in an incubator at 37° C., 5% CO₂, and 90% humidity. The growth medium was changed the day after seeding and every other day thereafter. Keratinocytes were used up to 18 population doublings. All cell preparations tested negative for mycoplasma, bacteria, yeast, and fungi. The condition of the skin keratinocytes was monitored with an inverted cell culture microscope (Westover Digital AMID Model 2000, Westover Scientific), controlled by a computer and images were captured using image acquisition software (Micron 2.0.0, Westover Scientific).

### III. Immunoblot Analysis

Protein lysates (20 μg of protein each) were boiled in 2× Laemmli buffer for 10 min and separated using a 10% gradient Tris-Glycine gel (or 4-20% gradient Tris-Glycine gel for caspase-8). Separated proteins were transferred to nitrocellulose membrane and the membranes were blocked in 2% BSA dissolved in (1×PBS) at room temperature (RT) for 15 min. Membranes were then incubated with P44/42 (Erk1/2) rabbit monoclonal antibody (1:1000), PhosphoP44/42 MAPK (Erk1/2) (Thr202/Tyr204) rabbit polyclonal antibody (1:1000), and anti-β-actin antibody (1:1000) for 1 hour at RT. Membranes were washed 3 times with standard immunoblot washing buffer and further incubated with alkaline phosphatase-conjugated secondary antibody (Jackson Immunoresearch Laboratories, West Grove, Pa.) at 1:10000 dilution for 45 min. Membrane fractions were washed 3 times with standard immunoblot washing buffer and developed using the 1-step NBT-BCIP substrate (Thermo Scientific, Rockford, Ill.). Signal intensities of the images were recorded in the ImageQuant RT-ECL (GE Life Sciences, Piscataway, N.J.) and densitometric quantification was performed using the IMAL software (Blanchette Rockefeller Neurosciences Institute, Morgantown, W. Va.). Intensities of Erk1/2 and p-Erk1/2 signals were normalized against (3-actin for each lane.

### IV. Cell Viability Test by MTT Assay

Cell viability assay was conducted by measuring 3-(4,5-dimethylthiazol-2-yl)-2,5-diphenyltetrazolium (MTT) bromide dye absorbance. After treatment, cells were labeled with MTT and dissolved by adding SDS-HCl solution to each well and mixing thoroughly with a pipette tip. Absorbance was read at 570 nm using a microplate reader. Data were presented in terms of relative cell viability.

### V. Human Skin Equivalents (HSEs)

HSEs have been used commercially as clinical skin substitutes (Orcel® from FortiCell Bioscience, Englewood Cliffs, N.J.; FDA approved in 2001/2008; Apligraf® from Genzyme Tissue Repair Corporation, Cambridge, Mass., FDA approved in 2007) and as in vitro models for skin biochemical tests and toxicity tests (Mertsching et al, 2008; Zhang and Michniak-Kohn, 2012). We engineered an HSE composed of both epidermal and dermal layers; keratinocytes and fibroblasts were utilized to prepare the bilayer structures (Shevchenko et al, 2010). Dermal fibroblasts were cultured on a 6-well collagen-coated surface (Biocoat Cell Environment, Becton Dickinson, Bedford, UK) or BD BIOCOAT™ plates (BD Biosciences, San Jose, Calif.). Fibroblast cells were maintained in DMEM with low glucose (Invitrogen) supplemented with 10% FBS, and were grown to 100% confluence. Culture medium was removed and the cells were washed three times with DMEM (without serum) to remove all growth factors. Previously cultured epidermal keratinocytes were added to the fibroblasts as the second layer (FIG. 1). The dermal fibroblast layer and epidermal keratinocyte layer were allowed to form HSE in keratinocyte basal medium supplemented with appropriate growth factors (keratinocyte basal medium-gold supplemented with BPE, 2 ml; hEGF, 0.5 ml; insulin, 0.5 ml; hydrocortisone, 0.5 ml; transferrin, 0.5 ml; epinephrine, 0.25 ml; GA-1000, 0.5 ml) in an incubator at 37° C., 5% CO₂, and 90% humidity. After 24 hours, the HSEs were treated with vehicle or increasing doses of bryostatin1. The condition of the HSEs was monitored with an inverted cell culture microscope (Westover Digital AMID Model 2000, Westover Scientific), controlled by a computer and images were captured via image acquisition software (Micron 2.0.0, Westover Scientific).

All of the references, patents and printed publications mentioned in the instant disclosure are hereby incorporated by reference in their entirety into this application.

We claim:

1. A method of improving one or more signs of aging comprising:
applying to a skin surface a topical composition comprising a therapeutically effective amount of a PKC activator, or an analog of a PKC activator, and a dermatologically acceptable carrier, wherein the composition is applied for a period of time sufficient to improve the appearance of one or more signs of aging skin.

2. The method of claim 1, wherein the composition further comprises at least one ingredient chosen from water, a solvent, a preservative, a surfactant, a gelling agent, and a pH balancer.

3. The method of claim 1, wherein the composition is in the form of a gel or a cream.

4. The method of claim 1, wherein the PKC activator is chosen from FGF-18, a macrocyclic lactone, benzolactam, a pyrrolidinone, bryolog, a fatty acid derivative, and a diacylglycerol derivative.

5. The method of claim 4, wherein the macrocyclic lactone is bryostatin.

6. The method of claim 5, wherein bryostatin is chosen from bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

7. The method of claim 4, wherein the bryolog is picolog.

8. The method of claim 1, wherein the PKC activator is provided in a range of about 0.3×10⁻⁷% to about 10% by weight of the topical composition.

9. The method of claim 1, wherein the PKC activator is provided in a range of about 0.01 nanomoles to about 10 micromoles per unit volume of the topical composition.

10. The method of claim 1, wherein the one or more signs of aging is chosen from age spots, wrinkle, stretch marks, increased skin transparency, acne, dry skin, and loss of elasticity.

11. A method for decreasing the formation of scar tissue following injury to the skin of a human subject, comprising:
applying a topical composition to the site of injury, the topical composition comprising a therapeutically effective amount of a PKC activator, or an analog of a PKC activator, and a dermatologically acceptable carrier.

12. The method of claim 11, wherein the composition is applied over a period of time from about 1 day to 30 days.

13. The method of claim 11, wherein the composition is applied once daily, twice daily, thrice daily or four times within a period of 24 hours.

14. The method of claim 11, wherein the composition further comprises an antibiotic agent.

15. A method for decreasing the formation scar tissue following surgery, comprising:
placing a topical composition comprising a therapeutically effective amount of a PKC activator, or an analog of a PKC activator onto a surface of a sterile mesh that is adapted for placement at a site of surgery or around a surgical incision, and
placing the mesh with the topical composition during surgery or after completing the surgical procedure.

16. The method of claim 15, wherein an analgesic drug is further placed on the surface of the sterile mesh.

17. A method for healing wounds or promoting wound-healing in a diabetic subject in need thereof, comprising:
applying to a wound of the subject a topical composition comprising a therapeutically effective amount of a PKC activator, or an analog of a PKC activator, and a dermatologically acceptable carrier, wherein the composition is applied for a period of time sufficient to heal the wound or promote healing of the wound.

18. The method of claim 17, wherein the composition further comprises an antibiotic agent.

19. A method for improving the acceptance of a clinical skin graft in a subject in need thereof, comprising
obtaining a donor skin as a graft tissue;
contacting the donor skin with a composition comprising a PKC activator, vitamins, amino acids, fibroblast growth factors and hormones for a period of 2-48 hours prior to use; and then
surgically grafting the donor skin to an area of a body of the subject with skin loss, wherein contact with the composition comprising a PKC activator, fibroblast growth factors and hormones reduces the risk of rejection by the subject receiving the graft.

20. The method of claim 19, wherein the fibroblast growth factors are chosen from bovine pituitary extract, human epithelial growth factor, insulin, transferrin, epinephrine, and hydrocortisone.

21. The method of claim 19, wherein the donor skin obtained as graft tissue is an autologous graft tissue, an allogeneic graft tissue, or an isogeneic graft tissue.

22. The method of claim 21, wherein the graft tissue is a split-thickness skin graft tissue or a full-thickness skin graft tissue.

23. The method of claim 19, wherein the subject is administered an immunosuppressive agent following surgical grafting of the donor skin.
